(19) United States
(12) Patent Application Publication
(10) Pub. No.: US 2008/0112966 A1
(43) Pub. Date: May 15, 2008

(54) Extracts and Methods Comprising Ganoderma Species

(76) Inventors: Robert T. Gow, Naples, FL (US); Dan Li, Singapore (SG); George W. Sypert, Naples, FL (US); Randall S. Alberte, Falmouth, ME (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/690,622

(22) Filed: Mar. 23, 2007

Related U.S. Application Data
(60) Provisional application No. 60/785,125, filed on Mar. 23, 2006.

(51) Int. Cl. A61K 36/074; C07J 9/00

(52) U.S. Cl. 424/195.15; 552/543; 552/546

(57) ABSTRACT

The present invention relates to extracts of ganoderma species plant material prepared by supercritical CO₂ extractions.

### RELATED APPLICATIONS

This application claims the benefit of priority to U.S. Provisional Patent Application Ser. No. 60/785,125, filed Mar. 23, 2006, which is hereby incorporated by reference in its entirety.

### FIELD OF INVENTION

The invention relates to extracts of ganoderma species, methods of preparing them using sequential extractions steps, and methods of treatment thereof.

### BACKGROUND OF THE INVENTION

Mushrooms are considered a special kind of food, particularly a “food delicacy” because of their unique texture and flavor. However, it was not until the 1900's, when antibiotics were obtained from the mold, Penicillin, that the potential medicinal value of fungi attracted the western scientific community. It has been shown that the chemical, biological, and biochemical properties of the chemical constituents of mushroom fruiting bodies are numerous with many physiological and medical benefits. The higher Basidiomycetes mushrooms have been used as herbal medicines throughout the world for thousands of years, particularly in Asia.

The ganoderma species, particularly G. lucidum (“Lingzhi” in China and “Reishi” or “Mannentake” in Japan) and G. tsuage, have been widely used for promoting health and longevity in China, Japan, and other Asian countries. Among cultivated mushrooms, ganoderma species are unique in that the pharmaceutical rather than the nutritional value is paramount. A wide variety of G. lucidum products are available in various forms, such as, powders, dietary supplements and beverages. These products are produced from different parts of the mushroom, including mycelia, fruiting body, and spores. However, the chemical constituent content of these products is suspect due to the large variation in the chemical constituents of the ganoderma species feedstock material. Like many botanical, the chemical constituents in the plant material is dependent on numerous variables including genetic drift, cultivation methods, temperature, pH, humidity, growth medium, substrates used, to list but a few of the variables.

The ganoderma species, family ganodermataceae, are polypore basidiomycetous fungi having a double-walled basidiospore. In all, 219 species within the family have been assigned to the genus ganoderma of which G. lucidum is the species type. Due to high phenotypic plasticity, morphological features for ganoderma systematics are thought to be of limited value in the identification of ganoderma species for extraction product feedstock. More recently, biochemical (triterpene constituents), genetic (mating studies) and molecular approaches (rDNA polymorphisms) have been used in ganoderma toxinomy.

Although traditional Chinese medicines (TCM) are used for their putative medicinal value, TCM is considered as a nutriceutical, and is categorized as a nutritional or dietary supplement in the United States, as defined by the Dietary Supplement Health and Education Act (DSHEA). One of the central questions for any therapy is the effective dose that produces a desired therapeutic action without harmful side effects. Ganoderma species have been used as a medicinal fungus for over 2000 years. However, there are no agreed upon standard formulations, chemical constituent compositions, or guidelines pertaining to its dosage, chemical composition, and formulation. Recommended dosages ranged from 0.5 gm to 30 gm of dried commercial extracts of G. lucidum fruiting body per day. There has been no significant toxicity reported even with very high levels of human consumption. Occasional mild digestive upset and skin rash in sensitive individuals have been reported. The toxic dose (TD) and lethal dose (LD) are very high with dosages as high as 5 g/kg administration to mice for 30 days and 38 g/kg injected as a single intra-peritoneal dose in laboratory animals are well tolerated. Therefore, the ganoderma species extraction products do not pose significant limitations for the clinical usage. Of importance is the determination of the effective and validation dose (ED) and scientific confirmation of ganoderma species chemical constituents' health benefits.

Like most mushrooms, ganoderma species are composed of about 90% water by weight. Based on the scientific literature, a summary of the G. lucidum chemical constituents by percent dry mass weight is listed the in Tables 1 and 2. One of the characteristics of the G. lucidum fruiting body is its bitterness that varies in degree depending on the strain, cultivation method, age, and a variety of other factors. The chemical constituents that convey this bitterness are the triterpenes and have been used as a marker for pharmacological evaluation of the extraction products. The two major known physiologically and medically active chemical constituents of the ganoderma species are the triterpenes and the polysaccharides.

TABLE 1
Chemical constituents of G. lucidum based on the literature.

| Principal Bio-actives* | % dry weight |
|---|---|
| Volatile/Essential Oil Chemical Compounds | 2-8% |
| Terpenoids* | |
| Triterpenes* (T) (>100 highly oxygenated lanostane- | |
| type triterpenoids) | |
| Ganoderic acids (GA) A, B, C, C1, C2, D . . . T | |
| Lucidenic acids (LA) A, B, C, C2, D, Di, K, E, E1, F, | |
| G, H, I, J, K | |
| Ganolucidic acids (GLA) C, D | |
| Ganoderiols (G) | |
| Lucidone (LC) A, D | |
| Lucidumols (LCM) A, B | |
| Ganodermenonol (G) | |
| Ganodermadiol (GD) | |
| Ganodermatriol (GT) | |
| Ganodermanondiol (GDD) | |
| Ganodermanontriol (GDT) | |
| Steroids | |
| Vitamins | |
| Phenols | |
| Nucleotides | |
| Proteins (Pr) | 7-8% |
| Glycoproteins | |
| Carbohydrates | 26-28% |
| Polysaccharides*(P) | |
| (Heteropolymers-glucose, xylose, mannose, glalactose, | |
| fucose, etc.) | |
| (β-D-glucans, particularly β-(1→3)-D-glucans) | |
| Ganoderans A, B, & C | |
| Fiber | 32-59% |
| Ash | 8-10% |
| Minerals | 10.2% |
| Germanium (Ge) | (489 μg/g) |

TABLE 2
Chemical composition of ganoderma lucidum fruit
body feedstock used in the present invention.

| Chemicals* | GL mushroom |
|---|---|
| Volatiles (%) | 1.2 |
| Tritepenoid (%) | 0.9 |
| Polysaccharide (%) | 1.59 |
| Protein (%) | |

*Volatile oil was estimated by highest yield of CO2 extraction at 70 C. and 500 bar. Tritepenoid were estimated by maximal methanol extraction. Polysaccharide and protein were estimated by water extract.

The terpenes are a class of naturally occurring compounds. Their carbon skeletons are composed of isoprene C5 units. Many are alkenes but may contain other functional groups, and many are cyclical. Some of the botanical terpenes have been found to possess properties such as anti-inflammatory, anti-cancer, hypolipidemic, and other health promoting activities. The triterpenes are a sub-class of the terpenes and have a basic skeleton of C30. In the ganoderma species, the chemical structure of the triterpenes is based on lanostane, a metabolite of lanosterol, the biosynthesis of which is based cyclization of squalene. Extraction of the triterpenes from ganoderma species is generally by solvent extraction using methanol, ethanol, acetone, chloroform, ether, or a mixture of these solvents. More than 100 triterpenes with known chemical composition and molecular configuration have been reported to occur in ganoderma species. Among them, the majority are found to be unique to ganoderma species. The large majority of the ganoderma triterpenes are ganoderic and lucidenic acids, but other triterpenens, such as ganoderals, ganoderiols, and ganodermic acids, have also been identified.

Botanical polysaccharides from a variety of plants have been reported to possess immune enhancement, anti-inflammatory, anti-ulcer, anti-viral, and anti-cancer effects. Ganoderma species are remarkable for producing a variety of high-molecular weight polysaccharides. These polyglycans are found in all parts of the mushroom as well as in all developments stages. Polysaccharides from ganoderma species have been extracted from the fruit body, mycelia, and spores. Moreover, exo-polysaccharides are produced by mycelia grown in fermenters. Glucose is the major sugar in ganoderma species polysaccharides. Ganoderma species, however, are heteropolymers that also contain xylose, mannose, and fucose in different configurations, including 1-3, 1-4, 1-6-linked beta, and alpha-D (or L)-polysaccharides. Polysaccharides are usually extracted with hot water followed by precipitation with alcohol. They can also be extracted with hot water and alkali. Complex purification steps can result in purified polysaccharide compounds such as the glucose polymer GL-1 (98% glucose). Polysaccharide compounds that have been isolated and partially characterized from ganoderma species include the Ganoderans A, B, and C. More recently, other ganoderma species polysaccharide compounds have been isolated. Some of these polysaccharide compounds have been shown to have significant immunological stimulating and anti-cancer activities.

Ganoderma species proteins, which are in lower amounts than other fungi, have also been reported to contribute to the medicinal activity of the ganoderma species chemical constituents. For instance, ganoderma species proteins may exhibit immunosuppressive activity.

In most medicinally valuable botanicals, the volatile oil and essential oil chemical constituents make major contributions to the bioactivity of the plant chemical constituents. However, these Ganoderm chemical constituents appear to have been ignored in the scientific literature.

The combination of putative health benefits without toxicity make ganoderma species chemical constituents desirable for the development of effective therapeutic extractions. Although ganoderma species extracts have been used for thousands of years as a treatment for various ailments, it is only in recent years that objective scientific studies of ganoderma species extracts and chemical constituents have been performed. To briefly summarize the therapeutic benefits of ganoderma species chemical constituents, recent scientific laboratory and clinical studies have demonstrated the following therapeutic effects of various chemical compounds, chemical fractions, and gross extraction products of ganoderma specie, particularly G. lucidum, including the following: immune enhancement (P, Pr, water extract-for abbreviation see Table 1) [1-4]: immuno-suppression, anti-transplant rejection, auto-immune disorders (Pr) [5,6]: anti-inflammatory, anti-arthritis, anti-rheumatoid, anti-lupus erythematosis, anti-allergy (T, GA, ethyl acetate extract, alcohol extract, water extract) [7-10]; anti-oxidant (T, P-T+P act synergistically, organic solvent extract, water extract) [9,11,12]; anti-platelet aggregation (GA, water soluble extract) [13,14]; hypoglycemic, anti-diabetic (P-Ganoderans A, B, & C, extract) [9,15]; anti-hypertensive (water soluble-ethanol insoluble extract, crude extract) [16,17]; anti-hypercholesterolemia (triterpenes, crude extract) [18]; prevention of cardiovascular diseases (T, P, crude extract) [5-18]; hepatoprotection (T, GA, P, water and water-ether extracts) [19,20]; anti-viral therapy, anti-herpes simplex, anti-HIV, anti-herpes zoster, anti-hepatitis B (P-protein bound polysaccharides, T, alcohol & water soluble extracts) [21-24]; anti-bacterial activity (T, P, alcohol and water extracts) [9,25]; and cancer prevention and treatment (P, T, hot-water & alcohol extract) [9, 26-28].

What is needed are novel and reproducible ganoderma extracts that combine purified essential oil, triterpene, protein, and polysaccharide chemical constituents that can be produced with standardized and reliable amounts of these synergistically acting physiologically and medically beneficial ganoderma species chemical constituents.

### SUMMARY OF THE INVENTION

In one aspect, the present invention relates to a ganoderma species extract comprising a fraction having a Direct Analysis in Real Time (DART) mass spectrometry chromatogram of any of FIGS. 6 to 29. In a further embodiment, the extract comprises a compound selected from the group consisting of an essential oil, a triterpene, a polysaccharide, and combinations thereof.

In a further embodiment, the essential oil is selected from the group consisting of 9,12-octadecadienoic acid, linoelaidic acid, n-hexadecanoic acid, octoanoic acid, tetradecanoic acid, pentadecanoic acid, 9-octadecenoic acid, octadecanoic acid, 2-propenoic acid, tridecyl ester, 1-undecanol, 1-dodecanol, 1-tetradecanol, 1-hexadecanol, 1-heptadecanol, 1-eicosanol, and combinations thereof. In a further embodiment, the amount of essential oil is greater than 8% by weight. In a further embodiment, the amount of essential oil is from 25% to 90% by weight. In a further embodiment, the amount of essential oil is from 50% to 90% by weight. In a further embodiment, the amount of essential oil is from 75% to 90% by weight.

In a further embodiment, the triterpene is selected from the group consisting of ganoderic acid, lucidenic acid, ganolucidic acid, ganoderiol, lucidone, lucidumol, ganodermenonol, ganodermadiol, ganodermatriol, ganodermanondiol, ganodermanontriol, and combinations thereof. In a further embodiment, the amount of triterpene is greater than 2% by weight. In a further embodiment, the amount of triterpene is from 25% to 90% by weight. In a further embodiment, the amount of triterpene is from 50% to 90% by weight. In a further embodiment, the amount of triterpene is from 75% to 90% by weight.

In a further embodiment, the polysaccharide is selected from the group consisting of glucose, arabinose, galactose, rhamnose, xylose uronic acid and combinations thereof. In a further embodiment, the amount of polysaccharide is greater than 15% by weight. In a further embodiment, the amount of polysaccharide is from 25% to 90% by weight. In a further embodiment, the amount of polysaccharide is from 50% to 90% by weight. In a further embodiment, the amount of polysaccharide is from 75% to 90% by weight.

In a further embodiment, the extract comprises an essential oil from 2% to 99% by weight, a triterpene from 5% to 88% by weight, and a polysaccharide from 2% to 95% by weight.

In another aspect, the present invention relates to a food or medicament comprising a ganoderma species extract of present invention.

In another aspect, the present invention relates to a method of preparing a ganoderma species extract having at least one predetermined characteristic comprising sequentially extracting a ganoderma species plant material to yield an essential oil fraction, a triterpene fraction, and a polysaccharide fraction by a) extracting a ganoderma species plant material by super critical carbon dioxide extraction to yield an essential oil fraction and a first residue; b) extracting the first residue from step a) by alcoholic extraction to yield the triterpene fraction and a second residue; and c) extracting the second residue from step b) by water extraction and precipitating the polysaccharide with alcohol to yield the polysaccharide fraction.

In a further embodiment, step a) comprises: 1) loading in an extraction vessel ground ganoderma species plant material; 2) adding carbon dioxide under supercritical conditions; 3) contacting the ganoderma species plant material and the carbon dioxide for a time; and 4) collecting an essential oil fraction in a collection vessel. In a further embodiment, the method further comprises the step of altering the essential oil chemical compound ratios by fractionating the essential oil fraction with a supercritical carbon dioxide fractional separation system. In a further embodiment, supercritical conditions comprise 60 bars to 800 bars of pressure at 35° C. to 90° C. In a further embodiment, supercritical conditions comprise 60 bars to 500 bars of pressure at 40° C. to 80° C. In a further embodiment, the time is 30 minutes to 2.5 hours. In a further embodiment, the time is 1 hour.

In a further embodiment, step b) comprises: 1) contacting the first residue from step a) with an alcoholic solvent for a time sufficient to extract triterpene chemical constituents; 2) purifying the triterpene chemical constituents using liquid-liquid solvent extraction processes. In a further embodiment, one solvent is chloroform and the other solvent is a saturated NaHCO₃ aqueous solution. In a further embodiment, the alcoholic solvent is ethanol. In a further embodiment, step 1) is carried out at 30° C. to 100° C. In a further embodiment, step 1) is carried out at 60° C. to 100° C. In a further embodiment, the time is 1-10 hours. In a further embodiment, the time is 1-5 hours. In a further embodiment, the time is 2 hours.

In a further embodiment, step c) comprises: 1) contacting either ganoderma species plant material or the second residue from step b) with water for a time sufficient to extract polysaccharides; and 2) precipitating the polysaccharides from the water solution by alcohol precipitation. In a further embodiment, the water is at 70° C. to 90° C. In a further embodiment, the water is at 80° C. to 90° C. In a further embodiment, the time is 1-5 hours. In a further embodiment, the time is 2-4 hours. In a further embodiment, the time is 2 hours. In a further embodiment, the alcohol is ethanol.

In another aspect, the present invention relates to a ganoderma species extract prepared by the methods of the present invention.

In another aspect, the present invention relates to a ganoderma species extract comprising ergosterol, ganolucidic acid A at 25 to 35% by weight of the ergosterol, ganolucidic acid B at 10 to 20% by weight of the ergosterol, and ganoderic acid H at 30 to 40% by weight of the ergosterol.

In another aspect, the present invention relates to a ganoderma species extract comprising ganoderic acid H and ganolucidic acid A at 25 to 35% by weight of the ganoderic acid H.

In another aspect, the present invention relates to a ganoderma species extract comprising ganoderic acid H, lucidenic acid B at 5 to 15% by weight of the ganoderic acid H, lucidenic acids A/N at 1 to 10% by weight of the ganoderic acid H, and ganolucidic acid A at 35 to 45% by weight of the ganoderic acid H.

In another aspect, the present invention relates to a ganoderma species extract comprising ganoderic acid H and ganoderal at 5 to 15% by weight of the ganoderic acid H.

In another aspect, the present invention relates to a ganoderma species extract comprising ganoderic acid H, ganolucidic acid A at 35 to 45% by weight of the ganoderic acid H, ganolucidic acid B at 10 to 20% by weight of the ganoderic acid H, and cerevisterol at 30 to 40% by weight of the ganoderic acid H.

In another aspect, the present invention relates to a ganoderma species extract comprising ganoderic acid H, ganolucidic acid B at 10 to 20% by weight of the ganoderic acid H, and ganoderal at 5 to 15% by weight of the ganoderic acid H.

In another aspect, the present invention relates to a ganoderma species extract comprising ganoderic acid H, ganolucidic acid B at 10 to 20% by weight of the ganoderic acid H, methoxycerevisterol at 20 to 30% by weight of the ganoderic acid H, and cerevisterol at 20 to 30% by weight of the ganoderic acid H.

In another aspect, the present invention relates to a ganoderma species extract comprising ergosterol, ganolucidic acid A at 30 to 40% by weight of the ergosterol, ganolucidic acid B at 5 to 15% by weight of the ergosterol, and ganoderic acid H at 65 to 75% by weight of the ergosterol.

In another aspect, the present invention relates to a ganoderma species extract comprising ganoderic acid H, ganolucidic acid B at 30 to 40% by weight of the ganoderic acid H, methoxycerevisterol at 40 to 50% by weight of the ganoderic acid H, and cerevisterol at 35 to 45% by weight of the ganoderic acid H.

In another aspect, the present invention relates to a ganoderma species extract comprising ergosterol, ganolucidic acids A/B at 1 to 10% by weight of the ergosterol, ganoderiol F at 1 to 10% by weight of the ergosterol, and lanosterol at 50 to 60% by weight of the ergosterol.

In another aspect, the present invention relates to a ganoderma species extract comprising ganoderic acid H, ganolucidic acid A at 60 to 70% by weight of the ganoderic acid H, ganolucidic acid B at 25 to 35% by weight of the ganoderic acid H, and lucidenic acids A/N at 10 to 20% by weight of the ganoderic acid H.

The extractions of the present invention are useful in providing physiological and medical effects including, but not limited to, immunological enhancement, immune suppression and anti-transplant rejection, anti-oxidant activity, anti-inflammatory activity, anti-arthritis, anti-rheumatoid, anti-auto-immune disease, anti-allergy, anti-platelet aggregation, hypoglycemic and anti-diabetes activity, anti-hypertensive, anti-hypercholesterolemia, prevention of cardiovascular disease and stroke, anti-mutagenic activity (cancer prevention), anti-carcinogenic activity (cancer therapy), anti-viral, anti-HIV, anti-herpes simplex, anti-herpes zoster, anti-hepatitis B, anti-bacterial activity, and hepato-protective and treatment for cirrhosis.

These embodiments of the disclosure, other embodiments, and their features and characteristics, will be apparent from the description, drawings and claims that follow.

### BRIEF DESCRIPTION OF THE DRAWINGS

FIG. 1 depicts an exemplary method for the preparation of the essential oil fraction.

FIG. 2 depicts an exemplary method for carrying out the ethanol leaching extraction.

FIG. 3 depicts an exemplary method for purification of the triterpene fraction.

FIG. 4 depicts an exemplary method for purification of the triterpene fraction.

FIG. 5 depicts an exemplary method for the water leaching process and polysaccharide precipitation.

FIG. 6 depicts AccuTOF-DART Mass Spectrum for ganoderma polysaccharide fraction from step 6 of the present methods (positive ion mode).

FIG. 7 depicts AccuTOF-DART Mass Spectrum for ganoderma polysaccharide fraction from step 6 of the present methods (negative ion mode).

FIG. 8 depicts AccuTOF-DART Mass Spectrum for ganoderma extract from red lingzhi young fruit (positive ion mode).

FIG. 9 depicts AccuTOF-DART Mass Spectrum for ganoderma essential oil extracted by SCCO₂ methods at 40° C. and 300 bar (postitive ion mode).

FIG. 10 depicts AccuTOF-DART Mass Spectrum for ganoderma essential oil extracted by SCCO₂ methods at 40° C. and 500 bar (postitive ion mode).

FIG. 11 depicts AccuTOF-DART Mass Spectrum for ganoderma essential oil extracted by SCCO₂ methods at 70° C. and 500 bar (postitive ion mode).

FIG. 12 depicts AccuTOF-DART Mass Spectrum for ganoderma essential oil extracted by SCCO₂ methods at 80° C. and 100 bar (postitive ion mode).

FIG. 13 depicts AccuTOF-DART Mass Spectrum for ganoderma essential oil extracted by SCCO₂ methods at 80° C. and 300 bar (postitive ion mode).

FIG. 14 depicts AccuTOF-DART Mass Spectrum for ganoderma essential oil extracted by SCCO₂ methods at 40° C. and 300 bar (postitive ion mode).

FIG. 15 depicts AccuTOF-DART Mass Spectrum for ganoderma essential oil extracted by SCCO₂ methods at 70° C. and 500 bar (postitive ion mode).

FIG. 16 depicts AccuTOF-DART Mass Spectrum for ganoderma essential oil extracted by SCCO₂ methods at 70° C. and 100 bar (postitive ion mode).

FIG. 17 depicts AccuTOF-DART Mass Spectrum for ganoderma ethanol crude extract (crude triterpenoid) from red lingzhi young fruit (positive ion mode).

FIG. 18 depicts AccuTOF-DART Mass Spectrum for final triterpenoid from red lingzhi young fruit (positive ion mode).

FIG. 19 depicts AccuTOF-DART Mass Spectrum for ganoderma extract from red lingzhi young fruit (negative ion mode).

FIG. 20 depicts AccuTOF-DART Mass Spectrum for ganoderma essential oil extracted by SCCO₂ methods at 40° C. and 300 bar (negative ion mode).

FIG. 21 depicts AccuTOF-DART Mass Spectrum for ganoderma essential oil extracted by SCCO₂ methods at 40° C. and 500 bar (negative ion mode).

FIG. 22 depicts AccuTOF-DART Mass Spectrum for ganoderma essential oil extracted by SCCO₂ methods at 70° C. and 500 bar (negative ion mode).

FIG. 23 depicts AccuTOF-DART Mass Spectrum for ganoderma essential oil extracted by SCCO₂ methods at 80° C. and 100 bar (negative ion mode).

FIG. 24 depicts AccuTOF-DART Mass Spectrum for ganoderma essential oil extracted by SCCO₂ methods at 80° C. and 300 bar (negative ion mode).

FIG. 25 depicts AccuTOF-DART Mass Spectrum for ganoderma essential oil extracted by SCCO₂ methods at 40° C. and 300 bar (negative ion mode).

FIG. 26 depicts AccuTOF-DART Mass Spectrum for ganoderma essential oil extracted by SCCO₂ methods at 70° C. and 500 bar (negative ion mode).

FIG. 27 depicts AccuTOF-DART Mass Spectrum for ganoderma essential oil extracted by SCCO₂ methods at 70° C. and 100 bar (negative ion mode).

FIG. 28 depicts AccuTOF-DART Mass Spectrum for ganoderma ethanol crude extract (crude triterpenoid) from red lingzhi young fruit (negative ion mode).

FIG. 29 depicts AccuTOF-DART Mass Spectrum for final triterpenoid from red lingzhi young fruit (negative ion mode).

### DETAILED DESCRIPTION OF THE INVENTION

Definitions

The articles “a” and “an” are used herein to refer to one or to more than one (i.e. to at least one) of the grammatical object of the article. By way of example, “an element” means one element or more than one element.

The term “ganoderma species” is also used interchangeably with lingzhi, reichi, or mannentake and means these plants, clones, variants, and sports, etc.

As used herein, the term “one or more compounds” means that at least one compound, such as 1-heptadecanol (a lipid soluble essential oil chemical constituent of ganoderma species), or ganoderic acid (a water and water-ethanol soluble triterpene of ganoderma species), or a water soluble-ethanol insoluble polysaccharide molecule of ganoderma species such as, but not limited to, Ganoderan A is intended, or that more than one compound, for example, 1-heptadecanol and Ganoderic acid A is intended. As known in the art, the term “compound” does not mean a single molecule, but multiples or moles of one or more compound. As known in the art, the term “compound” means a specific chemical constituent possessing distinct chemical and physical properties, whereas “compounds” refer to one or more chemical constituents.

As used herein, the term “fraction” means the extraction comprising a specific group of chemical compounds characterized by certain physical, chemical properties or physical or chemical properties.

As used herein, the term essential oil fraction comprises lipid soluble, water insoluble compounds obtained or derived from ganoderma species including, but not limited to, the chemical compounds classified as 1-heptadecanol, 2-propenoic acid, tridecyl ester, n-hexadecanoic acid, (Z)-9-octadecen-1-ol, 1-eicosanol, (Z,Z)-9,12-octadecadienoic acid, and linoelaidic acid.

As used herein, the term “triterpene fraction” comprises the water-soluble and ethanol soluble triterpene compounds obtained or derived from ganoderma species, further comprising, but not limited to, compounds such as Ganoderic acids, lucidenic acids, ganolucidic acids, ganoderiols, lucidone, and ganodermiatriol.

As used herein, the term “polysaccharide fraction” comprises water soluble-ethanol insoluble polysaccharide compounds obtained or derived from ganoderma species.

Other chemical constituents of ganoderma species may also be present in these extraction fractions.

As used herein, the term “purified” fraction means a fraction comprising a specific group of compounds characterized by certain physical-chemical properties or physical or chemical properties that are concentrated to greater than 50% of the fraction's chemical constituents. In other words, a purified fraction comprises less than 50% chemical constituent compounds that are not characterized by certain desired physical-chemical properties or physical or chemical properties that define the fraction.

As used herein, the term “profile” refers to the ratios by percent mass weight of the chemical compounds within an extraction fraction or to the ratios of the percent mass weight of each of the three ganoderma species fraction chemical constituents in a final ganoderma species extraction.

As used herein, “feedstock” generally refers to raw plant material, comprising whole plants alone, or in combination with on or more constituent parts or stages of a plant comprising fruit bodies, mycelia, and spores, wherein the plant or constituent parts may comprise material that is raw, dried, steamed, heated or otherwise subjected to physical processing to facilitate processing, which may further comprise material that is intact, cut, chopped, diced, milled, ground or otherwise processed to affected the size and physical integrity of the plant material. Occasionally, the term “feedstock” may be used to characterize an extraction product that is to be used as feed source for additional extraction processes.

As used herein, the term “ganoderma species constituents” shall mean chemical compounds found in ganoderma species and shall include all such chemical compounds identified above as well as other compounds found in ganoderma species, including but not limited to the essential oil chemical constituents, triterpenes, proteins, and polysaccharides.

Extractions

The present invention comprises extractions comprising one or more chemical constituent fractions found in ganoderma and related species. The invention also comprises ingestible products that comprise the extractions comprising ganoderma and related species extractions taught herein. For example, the present invention comprises extractions comprising a rapid dissolve tablet, comprising an ganoderma or related species extract wherein at least one of an essential oil fraction, an essential oil sub-fraction, a triterpene fraction, or a polysaccharide fraction has been substantially increased in weight percent amount in relation to the weight percent amount of that found in the native plant material or to that currently found in known ganoderma species extracts.

Essential Oil Fraction

Ganoderma essential oil with purity greater than 95% was extracted by supercritical carbon dioxide (SCCO2) extraction techniques. The highest extraction yield was found to be 1.22% at temperature of 70° C. and pressure of 500 bar. A total of 75 compounds were identified using gas chromatography-mass spectroscopy (GC-MS) analysis. The major compounds found in ganoderma essential oil are C11-C20 fatty acids. The most abundant ones are C18 fatty acid, 9,12-octadecadienoic acid (Z,Z)-(CAS: 60-33-3) (compound 59)and linoelaidic acid, (E,Z)-Isomer (compound 60), both of them are stereoisomer. Linoelaidic Acid, (E,Z)-Isomer is a doubly unsaturated fatty acid, occurring widely in plant glycosides. It is an essential fatty acid in mammalian nutrition and is used in the biosynthesis of prostaglandins and cell membranes.

The second abundant compound is the C16 saturated fatty acid n-hexadecanoic acid (CAS: 57-10-3) (compound 46). Other fatty acids include: Octoanoic acid (C8H1602, CAS: 124-07-2), Tetradecanoic acid (C14H28O2, CAS: 544-63-8), Pentadecanoic acid (C15H30O2, CAS: 1002-84-9-Octadecenoic (C18H34O2, CAS: 112-80-1) and Octadecanoic acid (C18H36O2, CAS: 57-11

The second major group of compounds are the alcohols, which include: 1-undecanol (C11H24O, CAS: 112-42-5), 1-dodecanol (C12H26O, CAS: 112-53-8), 1-tetradecanol (C14H30O, CAS: 112-72-1), 1-Hexadecanol (C16H34O, CAS: 36653-82-4), 1-Heptadecanol (C17H36O, CAS: 1454-85-9) and 1-Eicosanol (C20H42O, CAS: 629-96-9) et al. These aliphatic alcohols remain unchanged and didn't transform into esters.

By using supercritical carbon dioxide extraction, the chemistry of ganoderma essential oil was profiled and the summarized results are shown in Table 3.

TABLE 3
Chemical profile of ganoderma essential oil
obtained at different SCCO2 conditions.

| | | | | T = | | |
|---|---|---|---|---|---|---|
| | T = 40° C. | | | 70° C. | T = 80° C. | |
| | 100 | 300 | 500 | 500 | 100 | 300 |
| P (bar) | bar | bar | bar | bar | bar | bar |
| Alcohols | 16.61 | 21.54 | 14.46 | 10.37 | 19.97 | 36.23 |
| Fatty acids | 79.52 | 68.34 | 80.55 | 84.75 | 74.19 | 54.74 |
| Peak 59 + 60 | 69.95 | 58.94 | 76.01 | 72.76 | 68.8 | 41.06 |
| Esters | 2.77 | 5.97 | 2.82 | 2.55 | 4.07 | 5.27 |
| Aldehydes | 0.37 | 2.55 | 0.34 | 0.44 | 0.41 | 1.56 |
| Ratio of (peak 50 + | 88.0 | 86.2 | 94.4 | 85.9 | 92.7 | 75.0 |
| 60)/fatty acid | | | | | | |

Fatty acids can be profiled between 54% and 85%. In the total fatty acids, compound 59, 9,12-octadecadienoic acid (Z,Z)- and compound 60, linoelaidic acid account for 75%-95% by mass weight. Alcohols can be profiled between 10% and 36%. With respect to other minor compounds present in ganoderma essential oil, esters can be profiled between 2.5% and 6% and aldehydes can be profiled between 0.37% and 2.55%. Higher concentrations of fatty acids can be obtained at high pressure, and higher concentrations of fatty alcohols can be obtained at low pressure of 100 bar and high temperature of 80° C.

Triterpene Fraction

Ganoderma triterpenes were extracted using ethanol and purified by liquid-liquid purification by using solubility change between triterpene acids and their salts by pH changing. In the final purified triterpene fraction, total triterpenes purity increased to 87.5% from 0.6% in feedstock and 19.9% in ethanol crude extracts. Three commercially available triterpene HPLC reference standards, Ganoderic acid A, Ganoderic acid F and ganodermatiol only take up approximately 4% in total triterpenes of ganoderma.

Polysaccharide Fraction

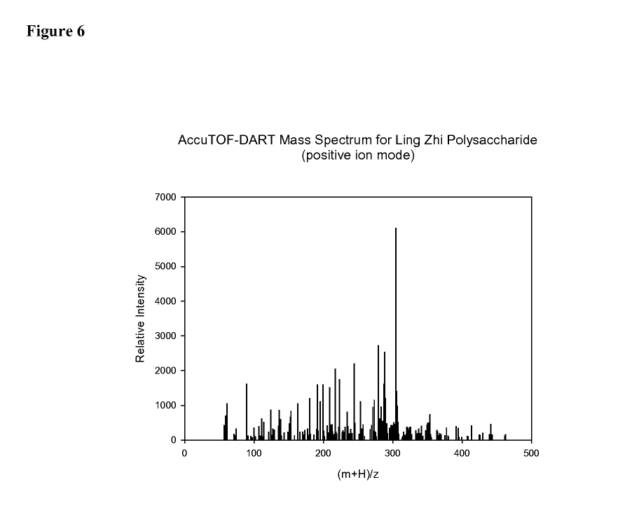

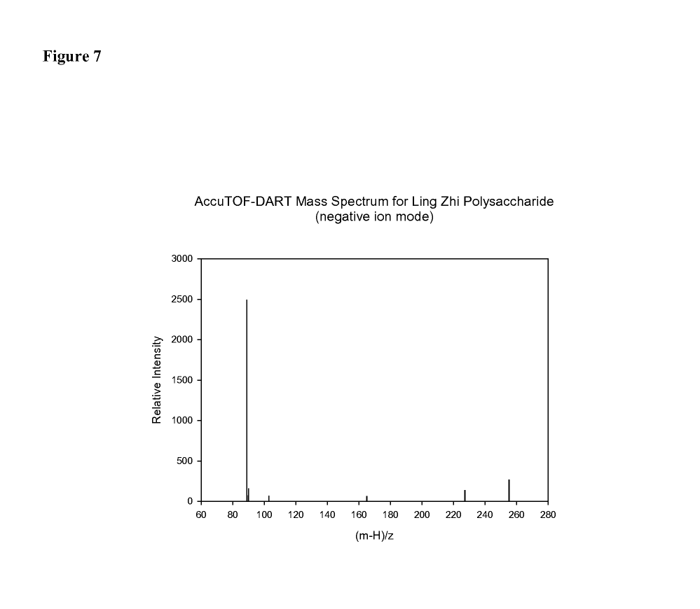

Ganoderma polysaccharides were extracted by distilled water and precipitated by 60-80% ethanol. The yield was 1.5%-2%. The purity of polysaccharides based on Dextran reference standards is 50%-80% depending on different molecular weights of Dextran. The average molecular weight of precipitated polysaccharide-glycoprotein was 953377, which is composing of different molecular weights of ganoderma polysaccharides and glycoproteins, in which 51% were polysaccharides and glycoproteins with high molecular weights of 1.6 M. The precipitated polysaccharide-glycoproteins were also characterized by Accu-TOF DART mass spectrum. The spectra are shown in FIGS. 6 and 7.

An embodiment of such extractions comprise predetermined concentrations of the extracted and purified chemical constituent fractions wherein the ganoderma species essential oil fraction/triterpen fraction, essential oil fraction/polysaccharide fraction, and triterpene fraction/polysaccharide fraction concentration (% dry weight) profiles (ratios) are greater or lesser than that found in the natural dried plant material or conventional ganoderma species extraction products. Alteration of the concentration relationships (chemical profiles) of the beneficial chemical constituents of the individual ganoderma species permits the formulation of unique or novel ganoderma species extract products designed for specific human conditions or ailments. For example, a novel and powerful ganoderma extraction for immune enhancement could have a greater purified polysaccharide fraction and a reduced essential oil fraction and triterpene fraction by % mass weight than that found in the ganoderma native plant material or conventional known extraction products. In contrast, a novel ganoderma extraction for anti-viral activity and anti-flu activity could have a greater purified triterpene fraction and a purified polysaccharide fraction and a reduced essential oil fraction by % mass weight than that found in the ganoderma native plant material or conventional known extraction products. Another example of a novel ganoderma extraction profile for anti-inflammatory activity could be an extraction profile with a greater purified essential oil fraction, purified triterpene fraction and purified polysaccharide fraction than that found in native ganoderma plant material or known conventional ganoderma extraction products.

A further embodiment of the invention is extractions comprising novel sub-fractions of the essential oil chemical constituents wherein the concentration of specific chemical groups such as, but not limited to, alcohols or fatty acids have their respective concentrations increased for decreased in novel extraction products.

Extractions Relative to Natural Ganoderma

Embodiments comprise extractions of ganoderma and related species having at least one of an essential oil, triterpene, or polysaccharide concentration that is in an amount greater than that found in the native ganoderma and related species plant material or currently available ganoderma species extract products. Embodiments also comprise extractions wherein one or more of the fractions, including essential oils, triterpenes, or polysaccharides, are found in a concentration that is greater than that found in native ganoderma species plant material. Embodiments also comprise extractions wherein one or more of the fractions, including essential oils, triterpenes, or polysaccharides, are found in a concentration that is less than that found in native ganoderma species. Known amounts of the bio-active chemical constituent fractions of the ganoderma species (Table 1) are used as an example of the present invention. For example, extractions of the present invention comprise fractions wherein the concentration of essential oils is from 0.001 to 80 times the concentration of native ganoderma species, and/or fractions where the concentration of triterpenes is from 0.001 to 100 times the concentration of native ganoderma species, and/or fractions where the concentration of polysaccharides is from 0.001 to 70 times the concentration of native ganoderma species. Extractions of the present invention comprise fractions wherein the concentration of essential oils is from 0.01 to 80 times the concentration of native ganoderma species, and/or fractions wherein the concentration of triterpenes is from 0.01 to 100 times the concentration of native ganoderma species, and/or fractions wherein the concentration of polysaccharides is from 0.01 to 70 times the concentration of native ganoderma species. Furthermore, extractions of the present invention comprise sub-fractions of the essential oil chemical constituents having at least one or more of chemical compounds present in the native plant material essential oil that is in amount greater or less than that found in native ganoderma plant material essential oil chemical constituents. For example, the ester, 2-propenoic acid, tridecyl ester, may have its concentration range from 0.22-2.53% by mass weight of an essential oil sub-fraction depending on the SCCO2 extraction conditions, a 12 fold increase range in concentration. In contrast, fatty acid, n-hexadecanoic acid, may have its concentration range from 4.00-9.86 by mass weight in an essential oil sub-fraction, a 2.5 fold range in concentration. Furthermore, the ratios of these two essential oil compounds may range from 1/15-1/3. As documented in Table 3, different essential oil sub-fractions may contain a widely different chemical constituents and chemical constituent ratios. Extractions of the present invention comprise fractions wherein the concentration of specific chemical compounds in such novel essential oil sub-fractions is either increase by about 1.1 to about 6 times or decreased by about 0.1 to about 6 times that concentration found in the native ganoderma essential oil chemical constituents.

For example, extractions of the present invention comprise fractions where the concentration of the essential oil chemical constituents is from 0.001 to 100 times the concentration of native ganoderma plant material, and/or fractions where the concentration of triterpenes is from 0.0001 to 100 times the concentration of native ganoderma plant material, and/or polysaccharides is from 0.001 to 100 times the concentration of native ganoderma plant material. In making a combined extraction, from about 0.001 mg to about 200 mg of an essential oil fraction can be used. Additionally, from about 0.001 mg to about 500 mg of a triterpene fraction can be used. Further, from about 0.001 mg to about 500 mg of the water-soluble ethanol insoluble polysaccharide fraction can be used.

Methods of Extraction

Methods of the present invention comprise providing novel ganoderma extractions for treatment and prevention of human disorders. For example, a novel ganoderma species extraction for immune enhancement activity may have an increased polysaccharide fraction concentration and reduced essential oil and triterpene fraction concentrations, by % weight, than that found in the ganoderma species native plant material or conventional known extraction products. A novel ganoderma species extraction for prevention and treatment of viral diseases may have an increased triterpene and polysaccharide fraction and a reduced essential oil fraction, by % weight, than that found in the native ganoderma species plant material or conventional known extraction products. Another example of a novel ganoderma species extraction for prevention and treatment of cancer comprises a fraction having an increased triterpene fraction concentration, an increased polysaccharide fraction, and an increased essential oil fraction than that found in native ganoderma species plant material or known conventional extraction products.

Additional embodiments comprise extractions comprising altered profiles (ratio distribution) of the chemical constituents of the ganoderma species in relation to that found in the native plant material or to currently available ganoderma species extract products. For example, the essential oil fraction may be increased or decreased in relation to the triterpene and/or polysaccharide concentrations. Similarly, the triterpenes or polysaccharides may be increased or decreased in relation to the other extract constituent fractions to permit novel constituent chemical profile extractions for specific biological effects.

The following methods as taught may be used individually or in combination with the disclosed method or methods known to those skilled in the art.

The starting material for extraction is plant material from one or more ganoderma species. The plant material may be the any portion of the plant, though the fruit body or mycelia are the most preferred starting material.

The ganoderma species plant material may undergo pre-extraction steps to render the material into any particular form, and any form that is useful for extraction is contemplated by the present invention. Such pre-extraction steps include, but are not limited to, that wherein the material is chopped, minced, shredded, ground, pulverized, cut, or torn, and the starting material, prior to pre-extraction steps, is dried or fresh plant material. A preferred pre-extraction step comprises grinding and/or pulverizing the ganoderma species plant material into a fine powder. The starting material or material after the pre-extraction steps can be dried or have moisture added to it. Once the ganoderma species plant material is in a form for extraction, methods of extraction are contemplated by the present invention.

Table 4 lists the principal beneficial bioactive chemical constituent fractions and some of the principal bioactive chemical compounds found in ganoderma species feedstock used in the present invention.

TABLE 4
Principle beneficial bioactive chemical constituents forund in ganoderma feedstock.

| | Ret | | | | | |
|---|---|---|---|---|---|---|
| Peak | time | | | | | |
| # | (min) | compound | structure | CAS# | Formula | Mw |
| 1 | 7.16 | 2-Heptenal (E)- | 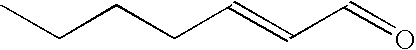 | 18829-55-5 | C7H12O | 112 |
| 2 | 9.63 | Methylene- butyrolactone | 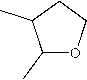 | 547-65-9 | C5H6O2 | 98 |
| 3 | 10.41 | 5-methyl-heptanol | 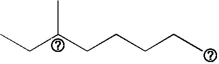 | 7212-53-5 | C8H18O | 130 |
| 4 | 11.90 | acetic acid, pentyl ester | 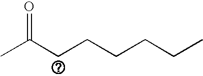 | 628-63-7 | C7H14O2 | 130 |
| 5 | 12.03 | Nonanal | 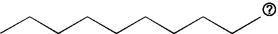 | 124-19-6 | C9H18O | 142 |
| 6 | 14.38 | Octanoic Acid | 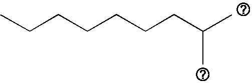 | 124-07-2 | C8H16O2 | 144 |
| 7 | 15.27 | trans-2,2- Dimethyl-3- heptene | 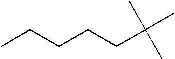 | 19550-75-5 | C9H18 | 126 |
| 8 | 17.18 | 3-methyl-5- undecene | 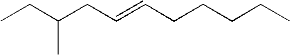 | 74630-67-4 | C12H24 | 168 |
| 9 | 17.50 | Benzene 1,3- bis(1,1- dimethylethyl)- | 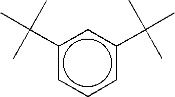 | 1014-60-4 | C14H22 | 190 |
| 10 | 17.70 | 2-Dodecenal (E)- | 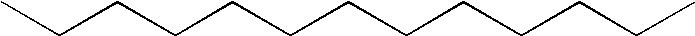 | 20407-84-5 | C12H22O | 182 |
| 11 | 19.02 | 2,4-Decadienal | 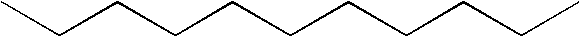 | 2363-88-4 | C10H16O | 152 |
| 12 | 19.71 | 2,2-dimethyl-3- decene | 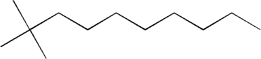 | 55499-02-0 | C12H24 | 168 |
| 13 | 20.04 | 1-dodecyn-4-ol | 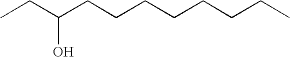 | 74646-36-9 | C12H22O | 182 |
| 14 | 20.52 | 2-isopropyl-5- methyl-1-heptanol | 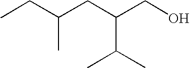 | 91337-07-4 | C11H24O | 172 |
| 15 | 20.97 | 1-dodecanol | 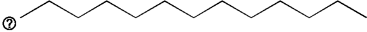 | 112-53-8 | C12H26O | 186 |
| 16 | 21.83 | propanoic acid, 2,2-dimethyl cyclohexyl ester | 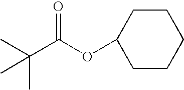 | 29878-49-7 | C11H20O2 | 184 |
| 17 | 22.65 | 2-undecanal | 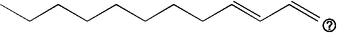 | 2463-77-6 | C11H20O | 168 |
| 18 | 27.56 | 1-undecanol | 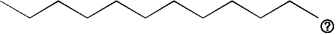 | 112-42-5 | C11H24O | 172 |
| 19 | 31.60 | 1-dodecanol | 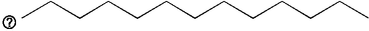 | 112-53-8 | C12H26O | 186 |
| 20 | 35.07 | 2-methyl-2- dodecanol | 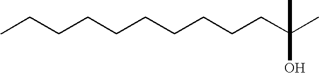 | 1653-37-8 | C13H28O | 200 |
| 21 | 35.17 | 3,5-bis(1,1- dimethylethyl)- phenol | 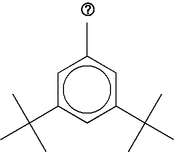 | 1138-52-9 | C14H22O | 206 |
| 22 | 37.38 | 1-tridenyn-4-ol | 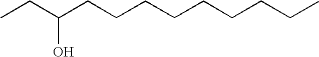 | 74646-37-0 | C13H24O | 196 |
| 23 | 38.01 | 1-dodecyn-4-ol | 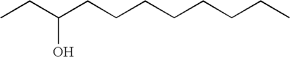 | 74646-36-9 | C12H22O | 182 |
| 24 | 40.74 | 2,6,10-trimethyl- dodecane | 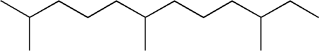 | 3891-98-3 | C15H32 | 212 |
| 25 | 41.52 | benzophenone | 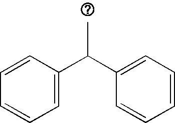 | 119-61-9 | C13H10O | 182 |
| 26 | 42.15 | 1-tetradecanol | 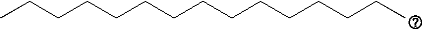 | 112-72-1 | C14H30O | 214 |
| 27 | 44.09 | dodecyl acrylate | 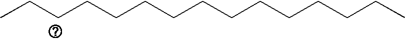 | 2156-97-0 | C15H28O2 | 240 |
| 28 | 44.79 | 2-Propenoic acid tridecyl ester | 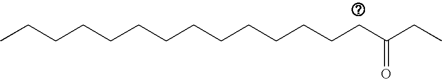 | 3076-08-4 | C16H30O2 | 254 |
| 29 | 45.11 | Hexadecane | 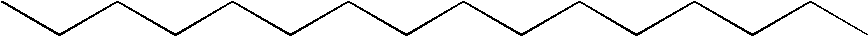 | 544-76-3 | C16H34 | 226 |
| 30 | 45.56 | Pentadecanal | 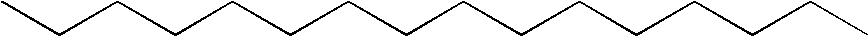 | 2765-11-9 | C15H30O | 226 |
| 31 | 47.73 | Tetradecanoic acid | 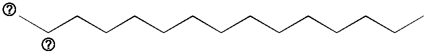 | 544-63-8 | C14H28O2 | 228 |
| 32 | 47.90 | 6-tridecanol | 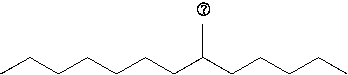 | 5770-06-3 | C13H28O | 200 |
| 33 | 48.66 | 2,6,11-trimethyl- dodecane | 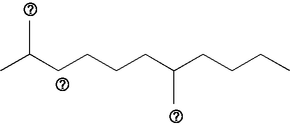 | 31295-56-4 | C15H32 | 212 |
| 34 | 48.97 | tetradecanal | 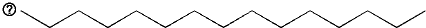 | 124-25-4 | C14H28O | 212 |
| 35 | 49.14 | 1,14- tetradecanediol | 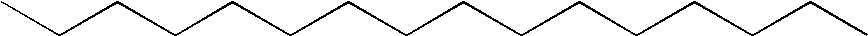 | 19812-64-7 | C14H30O2 | 230 |
| 36 | 49.68 | hexadecanal | 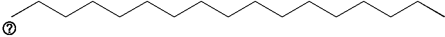 | 629-80-1 | C16H32O | 240 |
| 37 | 49.77 | unknown | | | | |
| 38 | 50.07 | Hexadecanal 2- methyl- | 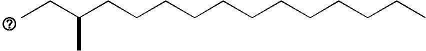 | 55019-46-0 | C17H34O | 254 |
| 39 | 50.54 | Pentadecanoic acid | 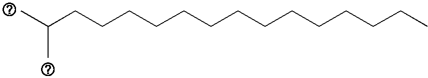 | 1002-84-2 | C15H30O2 | 242 |
| 40 | 50.69 | Phthalic acid diisobutyl ester | 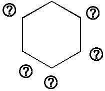 | 84-69-5 | C16H22O4 | 278 |
| 41 | 51.11 | 1-Heptadecanol | 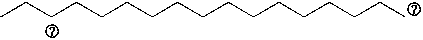 | 1454-85-9 | C17H36O | 256 |
| 42 | 52.23 | Octadecanal | 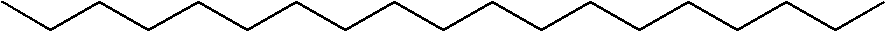 | 638-66-4 | C18H36O | 268 |
| 43 | 52.56 | 1-Hexadecanol | 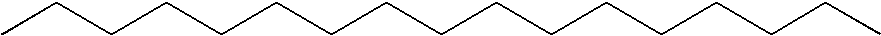 | 36653-82-4 | C16H34O | 242 |
| 44 | 52.93 | 9-Octadecenoic acid (Z)- | 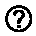 | 112-80-1 | C18H34O2 | 282 |
| 45 | 53.20 | 9-Octadecenoic acid (E)- | 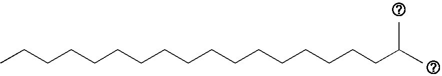 | 112-80-1 | C18H34O2 | 282 |
| 46 | 53.64 | n-Hexadecanoic acid | 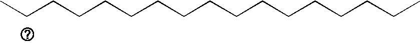 | 57-10-3 | C16H32O2 | 256 |
| 47 | 53.84 | dodecanoic acid 2-hexen-1-yl-ester | 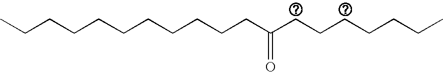 | 0-00-0 | C18H34O2 | 282 |
| 48 | 54.46 | n-butyl myrisate | 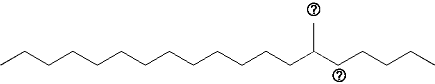 | 110-36-1 | C18H36O2 | 284 |
| 49 | 54.71 | 1.19-eicosadiene | 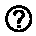 | 14811-95-1 | C20H38 | 278 |
| 50 | 55.32 | Octadecyl acetate | 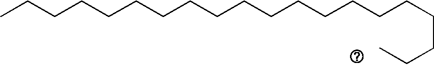 | 822-23-1 | C20H40O2 | 312 |
| 51 | 55.63 | 1-Eicosyne | 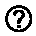 | 765-27-5 | C20H38 | 278 |
| 52 | 56.21 | Eicosene-1-ol cis- 9- | 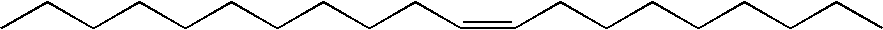 | 112248-30-3 | C20H40O | 296 |
| 53 | 56.36 | Z-8-Octadecen-1- ol acetate | 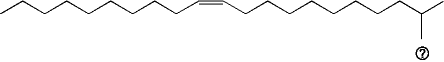 | 0-00-0 | C20H38O2 | 310 |
| 54 | 57.13 | 9-Octadecen-1-ol (Z)- | 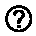 | 143-28-2 | C18H36O | 268 |
| 55 | 57.41 | 9-Octadecen-1-ol (E)- | 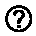 | 143-28-2 | C18H36O | 268 |
| 56 | 58.21 | 1-Eicosanol | 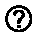 | 629-96-9 | C20H42O | 298 |
| 57 | 58.73 | dihydrofamesyl propanoate | 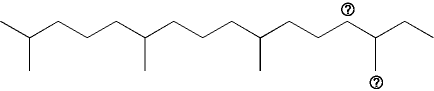 | 0-00-0 | C18H32O2 | 280 |
| 58 | 59.02 | phytol | 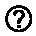 | 150-86-7 | C20H40O | 296 |
| 59 | 60.77 | 9,12- octadecadienoic acid (Z,Z)- | 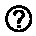 | 60-33-3 | C18H32O2 | 280 |
| 60 | 61.11 | Linoelaidic acid | 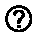 | 60-33-3 | C18H32O2 | 280 |
| 61 | 62.28 | Octadecanoic acid | 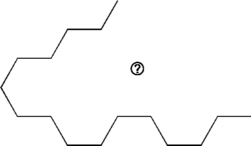 | 57-11-4 | C18H36O2 | 284 |
| 62 | 63.67 | Hexadecanoic acid butyl ester | 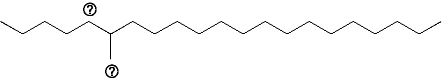 | 111-06-8 | C20H40O2 | 312 |
| 63 | 66.67 | (R)-(-)-(Z)-14- methyl-8- hexadecen-1-ol | 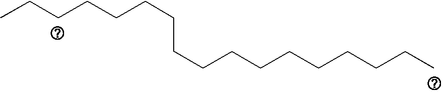 | 30689-78-2 | C17H34O | 254 |
| 64 | 67.02 | 3-heptadecanol | 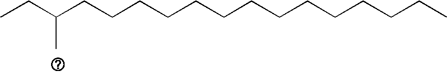 | 83543-30-5 | C17H36O | 256 |
| 65 | 68.50 | 9-octadecenal | 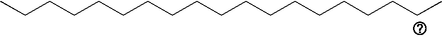 | 5090-41-5 | C18H34O | 266 |
| 66 | 70.50 | 9-eicosene (E)- | | 74685-29-3 | C20H40 | 280 |
| 67 | 71.26 | 2-Nonadecanone | 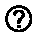 | 629-66-3 | C19H38O | 282 |
| 68 | 71.45 | (Z)-Phytol | 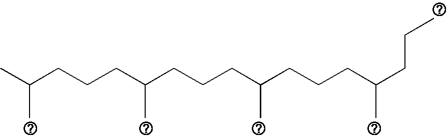 | | C20H40O | 296 |
| 69 | 73.00 | 3,7,11,15- tetramethyl, 2- hexadecen-1-ol | 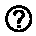 | 150-86-7 | C20H40O | 296 |
| 70 | 73.25 | 9- octadecanamide (Z)- | 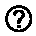 | 301-02-0 | C18H35NO | 281 |
| 71 | 74.35 | Cyclohexane- carboxamide, N-decyl-N-methyl- | 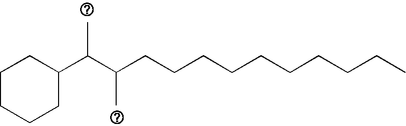 | | C18H35NO | 281 |
| 72 | 74.62 | Octadecanoic acid butyl ester | 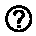 | 123-95-5 | C22H44O2 | 340 |
| 73 | 76.08 | Octadecanal | 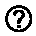 | 638-66-4 | C18H36O | 268 |
| 74 | 76.52 | 9-Octadecene- 1,12-diol | 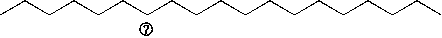 | 7706-08-3 | C18H36O2 | 294 |
| 75 | 76.78 | Propionic acid, 3- tetradecyloxy- methyl ester | 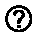 | 6064-97-7 | C18H36O3 | 300 |

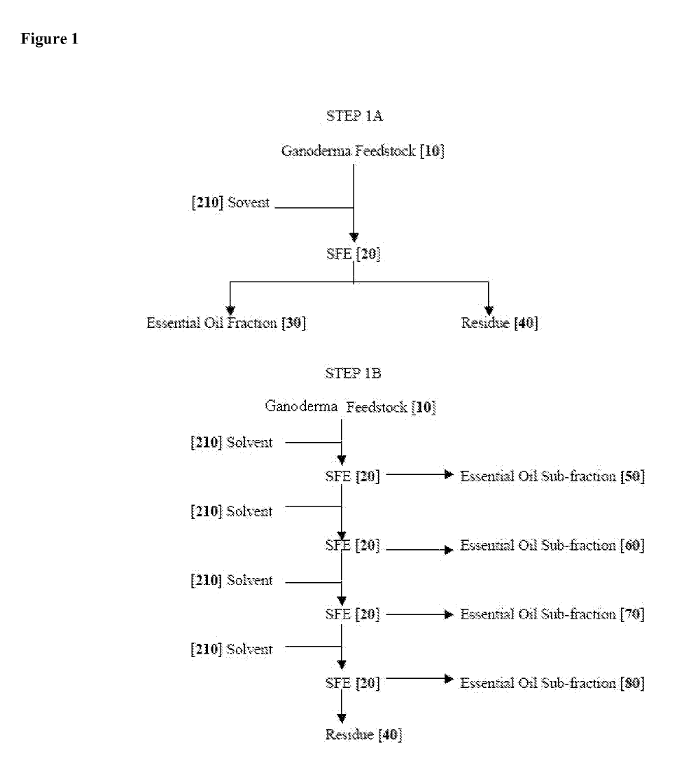

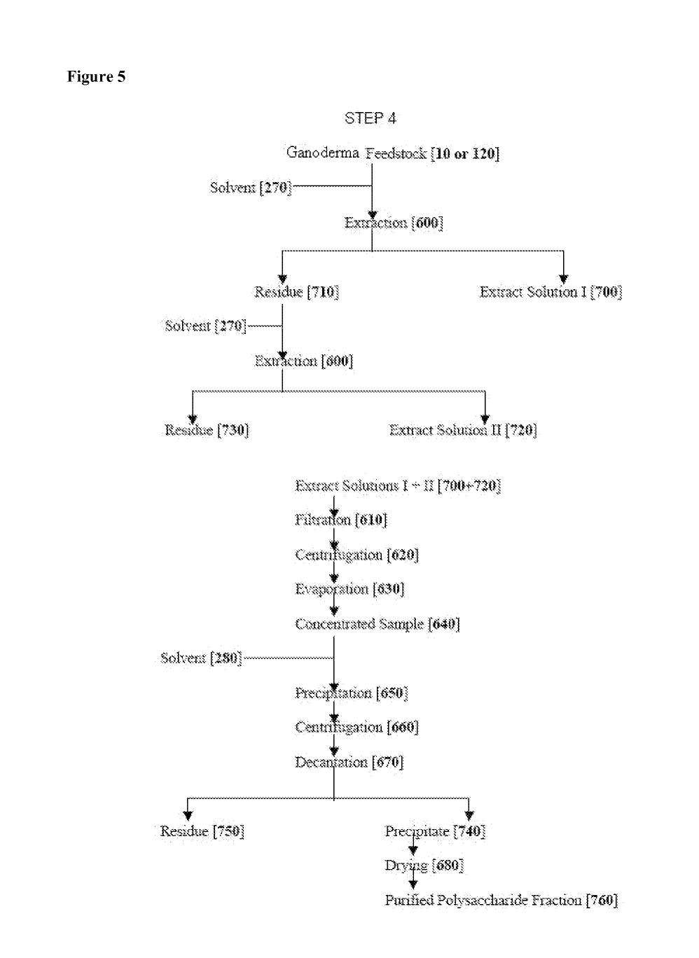

Methods of extraction of the present invention comprise processes disclosed herein. In general, methods of the present invention comprise, in part, methods wherein ganoderma species plant material is extracted using supercritical fluid extraction (SFE) with carbon dioxide as the solvent (SCCO₂) that is followed by one or more solvent extraction steps, such as, but not limited to, water, hydroalcoholic, and affinity polymer absorbent extraction processes. Additional other methods contemplated for the present invention comprise extraction of ganoderma species plant material using other organic solvents, refrigerant chemicals, compressible gases, sonification, pressure liquid extraction, high speed counter current chromatography, molecular imprinted polymers, and other known extraction methods. Such techniques are known to those skilled in the art. In one aspect, extractions of the present invention may be prepared by a method comprising the steps depicted schematically in FIGS. 1-5.

The invention includes processes for concentrating (purifying) and profiling the essential oil and other lipid soluble compounds from ganoderma plant material using SCCO2 technology. The invention includes the fractionation of the lipid soluble chemical constituents of ganoderma into, for example, an essential oil fraction of high purity (high essential oil chemical constituent concentration). Moreover, the invention includes a SCCO2 process wherein the individual chemical constituents within an extraction fraction may have their chemical constituent ratios or profiles altered. For example, SCCO2 fractional separation of the chemical constituents within an essential oil fraction permits the preferential extraction of certain essential oil compounds relative to the other essential oil compounds such that an essential oil extract sub-fraction can be produced with a concentration of certain compounds greater than the concentration of other compounds. Extraction of the essential oil chemical constituents of the ganoderma species with SCCO2 as taught in the present invention eliminates the use of toxic organic solvents and provides simultaneous fractionation of the extracts. Carbon dioxide is a natural and safe biological product and an ingredient in many foods and beverages.

A schematic diagram of the methods of extraction of the biologically active chemical constituents of Ligusticum is illustrated in FIGS. 1-5. The extraction process is typically, but not limited to, 4 steps. For reference in the text, when the bold number appears in brackets [x], the numbers refers to the numbers in FIGS. 1-5. The analytical methods used in the extraction process are presented in the Exemplification section.

Step 1: Supercritical Fluid Carbon Dioxide Extraction of Ganoderma Essential Oil

Due to the hydrophobic nature of the essential oil, non-polar solvents, including, but not limited to SCCO₂, hexane, petroleum ether, and ethyl acetate may be used for this extraction process. Since some of the components of the essential oil are volatile, steam distillation may also be used as an extraction process.

A generalized description of the extraction of the essential oil chemical constituents from the rhizome of the ganoderma species using SCCO2 is diagrammed in FIG. 1-Step 1A and 1B. The feedstock [10] is dried ground ganoderma species fruit body (about 140 mesh). The extraction solvent [210] is pure carbon dioxide. Ethanol may be used as a co-solvent. The feedstock is loaded into a into a SFE extraction vessel [20]. After purge and leak testing, the process comprises liquefied CO2 flowing from a storage vessel through a cooler to a CO2 pump. The CO2 is compressed to the desired pressure and flows through the feedstock in the extraction vessel where the pressure and temperature are maintained at the desired level. The pressures for extraction range from about 60 bar to 800 bar and the temperature ranges from about 35° C. to about 90° C. The SCCO2 extractions taught herein are preferably performed at pressures of at least 100 bar and a temperature of at least 35° C., and more preferably at a pressure of about 60 bar to 500 bar and at a temperature of about 40° C. to about 80° C. The time for extraction for a single stage of extraction range from about 30 minutes to about 2.5 hours, to about 1 hour. The solvent to feed ratio is typically about 60 to 1 for each of the SCCO2 extractions. The CO2 is recycled. The extracted, purified, and profiled essential oil chemical constituents [30] are then collected a collector or separator, saved in a light protective glass bottle, and stored in a dark refrigerator at 4° C. The ganoderma feedstock [10] material may be extracted in a one step process (FIG. 1, Step 1A) wherein the resulting extracted and purified ganoderma essential oil fraction [30] is collected in a one collector SFE or SCCO2 system [20] or in multiple stages (FIG. 1, Step 1B) wherein the extracted purified and profiled ganoderma essential oil sub-fractions [50, 60, 70, 80] are separately and sequentially collected in a one collector SFE system [20]. Alternatively, as in a fractional SFE system, the SCCO2 extracted ganoderma feedstock material may be segregated into collector vessels (separators) such that within each collector there is a differing relative percentage essential oil chemical constituent extraction (profile) in each of the purified essential oil sub-fractions collected. The residue (remainder) [40] is collected, saved and used for further processing to obtain purified fractions of the ganoderma species triterpenes and polysaccharides. An embodiment of the invention comprises extracting the ganoderma species feedstock material using multi-stage SCCO2 extraction at a pressure of 60 bar to 500 bar and at a temperature between 35° C. and 90° C. and collecting the extracted ganoderma material after each stage. A second embodiment of the invention comprises extracting the ganoderma species feedstock material using fractionation SCCO2 extraction at pressures of 60 bar to 500 bar and at a temperature between 35° C. and 90° C. and collecting the extracted ganoderma material in differing collector vessels at predetermined conditions (pressure, temperature, and density) and predetermined intervals (time). The resulting extracted ganoderma purified essential oil sub-fractions from each of the multi-stage extractors or in differing collector vessels (fractional system) can be retrieved and used independently or can be combined to form one or more ganoderma essential oil extractions comprising a predetermined essential oil chemical constituent concentration that is higher or lower than that found in the native plant material or in conventional ganoderma extraction products. Typically, the total yield of the essential oil fraction from ganoderma species using a single step maximal SCCO2 extraction is about 1.8% (>95% of the essential oil chemical constituents) by % weight having an essential oil chemical constituent purity of greater than 95% by mass weight of the extract. Examples as well as the results of such extraction processes are found below and in Tables 5 and 6. The procedure can be found in Example 1.

TABLE 5
GC-MS Peak area percentage of ganoderma lucidum
extract using SCCO2 at different conditions.

| Peak | ret time | compound | T = 40 C. | | | T = 80 C. | | T = 70 C. |
|---|---|---|---|---|---|---|---|---|
| # | (min) | property | 100 bar | 300 bar | 500 bar | 100 bar | 300 bar | 500 bar |
| 1 | 7.16 | aldehyde | | 0.54 | | | | 0.02 |
| 2 | 9.63 | lactones | | | | 0.08 | 0.13 | |
| 3 | 10.41 | alcohol | | 0.06 | | | | |
| 4 | 11.90 | ester | | | | | 0.06 | 0.03 |
| 5 | 12.03 | aldehyde | 0.04 | 0.08 | | | 0.13 | 0.02 |
| 6 | 14.38 | acid | | 0.16 | | | | 0.02 |
| 7 | 15.27 | alkene | | | | | | |
| 8 | 17.18 | alkene | 0.03 | 0.14 | | | 0.05 | |
| 9 | 17.50 | aromaric | 0.26 | 0.3 | 0.49 | 0.27 | 0.42 | 0.06 |
| | | compound | | | | | | |
| 10 | 17.70 | aldehyde | | 1.08 | | | | 0.07 |
| 11 | 19.02 | aldehyde | | 0.51 | | | | 0.03 |
| 12 | 19.71 | alkene | 0.05 | 0.07 | 0.16 | 0.05 | 0.08 | |
| 13 | 20.04 | alcohol | 0.07 | 0.63 | 0.2 | 0.09 | 0.12 | 0.07 |
| 14 | 20.52 | alcohol | 0.06 | 0.07 | 0.27 | 0.05 | 0.12 | |
| 15 | 20.97 | alcohol | | 0.05 | 0.1 | 0.11 | | 0.12 |
| 16 | 21.83 | ester | | | | | | |
| 17 | 22.65 | aldehyde | | | | | | |
| 18 | 27.56 | alcohol | | | | 0.03 | 0.07 | |
| 19 | 31.60 | alcohol | 0.21 | 0.4 | 0.47 | 0.28 | 0.51 | 0.11 |
| 20 | 35.07 | alcohol | 0.06 | 0.06 | 0.28 | 0.17 | 0.15 | 0.18 |
| 21 | 35.17 | phenol | | | | | | |
| 22 | 37.38 | alcohol | | | | | 0.07 | |
| 23 | 38.01 | alcohol | | | | | 0.07 | |
| 24 | 40.74 | alkane | 0.06 | | 0.1 | | 0.07 | |
| 25 | 41.52 | aromatic | | 0.11 | | | | 0.06 |
| 26 | 42.15 | alcohol | 0.08 | | 0.16 | 0.06 | 0.1 | |
| 27 | 44.09 | ester | 0.07 | 0.2 | | 0.15 | 0.22 | 0.07 |
| 28 | 44.79 | ester | 0.3 | 2.53 | 0.36 | 0.22 | 0.38 | 0.68 |
| 29 | 45.11 | alkane | 0.05 | 0.15 | 0.19 | 0.09 | 0.11 | 0.05 |
| 30 | 45.56 | aldehyde | 0.03 | 0.08 | | | 0.09 | 0.08 |
| 31 | 47.73 | fatty acid | | 0.1 | | | | 0.27 |
| 32 | 47.90 | alcohol | | | | | | |
| 33 | 48.66 | decane | | 0.08 | 0.2 | 0.11 | 0.09 | |
| 34 | 48.97 | aldehyde | | 0.07 | | | | |
| 35 | 49.14 | alcohol | | 0.07 | | 0.1 | 0.07 | 0.03 |
| 36 | 49.68 | aldehyde | | | 0.14 | 0.08 | 0.16 | |
| 37 | 49.77 | | 0.23 | 0.45 | 0.29 | 0.28 | 0.21 | 0.44 |
| 38 | 50.07 | aldehyde | | | 0.2 | | | 0.01 |
| 39 | 50.54 | fatty acid | 0.17 | 0.18 | | 0.09 | 0.15 | 0.39 |
| 40 | 50.69 | ester | 0.1 | 0.1 | | 0.07 | 0.06 | 0.09 |
| 41 | 51.11 | alcohol | 5.35 | 7.44 | 1.62 | 6.76 | 9.07 | 2.70 |
| 42 | 52.23 | aldehyde | 0.05 | 0.04 | | 0.04 | 0.08 | 0.05 |
| 43 | 52.56 | alcohol | | 0.03 | | | 0.11 | 0.04 |
| 44 | 52.93 | fatty acid | 0.14 | 0.08 | | | 0.08 | 0.23 |
| 45 | 53.20 | fatty acid | 0.2 | 0.09 | | | | 0.34 |
| 46 | 53.64 | fatty acid | 8.33 | 8.3 | 4.00 | 4.61 | 10.39 | 9.86 |
| 47 | 53.84 | ester | | | | | | |
| 48 | 54.46 | ester | | | 0.18 | 0.08 | 0.07 | |
| 49 | 54.71 | alkene | 0.05 | 0.08 | 0.16 | 0.07 | 0.13 | 0.22 |
| 50 | 55.32 | ester | | | | | | 0.03 |
| 51 | 55.63 | alkene | | | | | 0.09 | 0.04 |
| 52 | 56.21 | alcohol | 0.06 | | 0.19 | 0.09 | 0.23 | |
| 53 | 56.36 | ester | | | | | | 0.25 |
| 54 | 57.13 | alcohol | 5.59 | 6.98 | 3.13 | 6.91 | 13.44 | 3.75 |
| 55 | 57.41 | alcohol | 0.77 | 0.91 | 0.35 | 0.78 | 2.07 | 0.66 |
| 56 | 58.21 | alcohol | 4.13 | 4.55 | 2.58 | 3.99 | 8.84 | 2.29 |
| 57 | 58.73 | ester | | | | | | 0.03 |
| 58 | 59.02 | alcohol | | | | | 0.12 | |
| 59 | 60.77 | fatty acid | 31.11 | 22.99 | 29.4 | 26.94 | 11.22 | 38.23 |
| 60 | 61.11 | fatty acid | 38.84 | 35.95 | 46.61 | 41.86 | 29.84 | 34.53 |
| 61 | 62.28 | fatty acid | 0.73 | 0.49 | 0.54 | 0.69 | 3.06 | 0.88 |
| 62 | 63.67 | ester | 0.68 | 0.46 | 1.88 | 0.89 | 1.07 | 0.36 |
| 63 | 66.67 | alcohol | | | | | 0.17 | |
| 64 | 67.02 | alcohol | 0.17 | 0.14 | 0.22 | 0.19 | 0.37 | 0.06 |
| 65 | 68.50 | aldehyde | 0.2 | 0.15 | | 0.1 | 0.77 | 0.16 |
| 66 | 70.50 | alkene | | | 0.13 | 0.16 | | 0.3 |
| 67 | 71.26 | alkehyde | | | | 0.19 | 0.18 | |
| 68 | 71.45 | alcohol | | | 4.53 | | 0.25 | |
| 69 | 73.00 | alcohol | | | | | 0.09 | |
| 70 | 73.25 | amide | | | | | 0.24 | 0.08 |
| 71 | 74.35 | amide | | | | | 0.08 | |
| 72 | 74.62 | ester | 1.35 | 2.36 | | 2.15 | 2.43 | 0.79 |
| 73 | 76.08 | aldehyde | 0.05 | | | | 0.15 | |
| 74 | 76.52 | alcohol | 0.06 | 0.15 | 0.36 | 0.36 | 0.19 | 0.36 |
| 75 | 76.78 | ester | 0.27 | 0.32 | 0.4 | 0.51 | 0.98 | 0.22 |
| | | summation | 100 | 99.78 | 99.89 | 99.75 | 99.5 | 99.36 |
| | | alcohol | 16.6 | 21.5 | 14.5 | 20.0 | 36.2 | 10.4 |
| | | fatty acid | 79.52 | 68.34 | 80.55 | 74.19 | 54.74 | 84.75 |
| | | 50 + 60 | 69.95 | 58.94 | 76.01 | 68.8 | 41.06 | 72.76 |
| | | ester | 2.77 | 5.97 | 2.82 | 4.07 | 5.27 | 2.55 |
| | | aldehyde | 0.37 | 2.55 | 0.34 | 0.41 | 1.56 | 0.44 |

The effect of temperature on total extraction yield depends on the system pressure; at low pressure of 100 bar, the extraction yield is decreased as temperature is increased. This finding is attributed to the large change in density when pressure is manipulated near the solvent critical point (density of CO2 at 40 C is 0.64 g/cc and density of CO2 at 80 C is 0.227 g/cc). At higher pressures of 300 bar and 500 bar, on the other hand, the extraction yield is increased as temperature is increased. This finding is attributed temperature effect on vapor pressure of solute since CO2 's density doesn't change very much by temperature.

In the experiment range investigated, it can be clearly noted that for ganoderma mushroom system, density and pressure do not appear to have much effect on extraction yield. However, temperature has a substantial effect. Both pressure and temperature have an effect on extraction kinetics. An increase in temperature promotes an enhancement in vapor pressure of the compounds favoring the extraction. Additionally, the increase in diffusion coefficient and the decrease in solvent viscosity also help the compounds extraction from the herbaceous porous matrix as the temperature and pressure are increased to a higher value. In conclusion, high temperature and pressure should be used for maximal SCCO2 extraction from both kinetics and yield standpoint.

As can be noted from Tables 4 and 5, the major compounds found in ganoderma species fruit body feedstock are C11-C20 fatty acids. The most abundant ones are the higher alcohol C18 fatty acids, 9,12-octadecandienoic acid (Z, Z)- and linoelaidic acid (E, Z)-. Both are sterioisomers. Linioelaidic acid (E, Z)-isomer is a doubly unsaturated fatty acid, occurring widely in plant glycosides. The second major group of compounds found in the essential oil fractions is alcohols. The most abundant of these compounds are the higher C17, C18 and C20 alcohols. These aliphatic alcohols remained unchanged with extraction and did not transform into esters. A high purity of volatile oil compounds are present in SCCO2 essential oil extract fraction of ganoderma species feedstock material. Moreover, ganoderma species essential oil extract fractions may be profiled using SCCO2 (Table 3) For example, higher concentrations of the alcohols may be obtained at higher extraction temperatures such as 80 ::C and low pressures such as 100 bar. In contrast, higher concentrations of C18 fatty acid isomers can be obtained at temperatures of 40-70° C. and high pressure such as 500 bar.

Ganoderma species SCCO2 extraction yield was about 0.6-1.2% by mass weight of the feedstock at temperatures of 40-80° C. and pressures of 100-500 bar with a solvent/feed (S/F) ratio of 180 (Table 6).

TABLE 6
Influence of temperature and pressure on SCCO2 essential
oil extraction yield (by % mass weight of the feedstock)
at different extraction time.

| | T = 40° C. | | | T = 70° C. | T = 80° C. | |
|---|---|---|---|---|---|---|
| P (bar) | 100 | 300 | 500 | 500 | 100 | 300 |
| Den(g/cc) | 0.640 | 0.915 | 0.996 | 0.909 | 0.227 | 0.751 |
| t = 5 min | 0.15 | | 0.28 | | 0.33 | 0.64 |
| 10 min | 0.20 | 0.40 | 0.50 | 0.57 | 0.49 | 0.64 |
| 15 min | 0.45 | | 0.68 | | 0.66 | 0.67 |
| 20 min | 0.50 | 0.57 | 0.80 | 0.79 | 0.68 | 0.68 |
| 30 min | 0.69 | 0.64 | 0.84 | 0.96 | 0.69 | 0.77 |
| 60 min | 0.82 | 0.78 | 0.85 | 1.22 | 0.71 | 0.82 |
| 90 min | 0.87 | 0.78 | 0.85 | 1.22 | 0.71 | 0.83 |

Step 2. Ethanol Leaching Process for Extraction of Crude Triterpenoid Fraction.

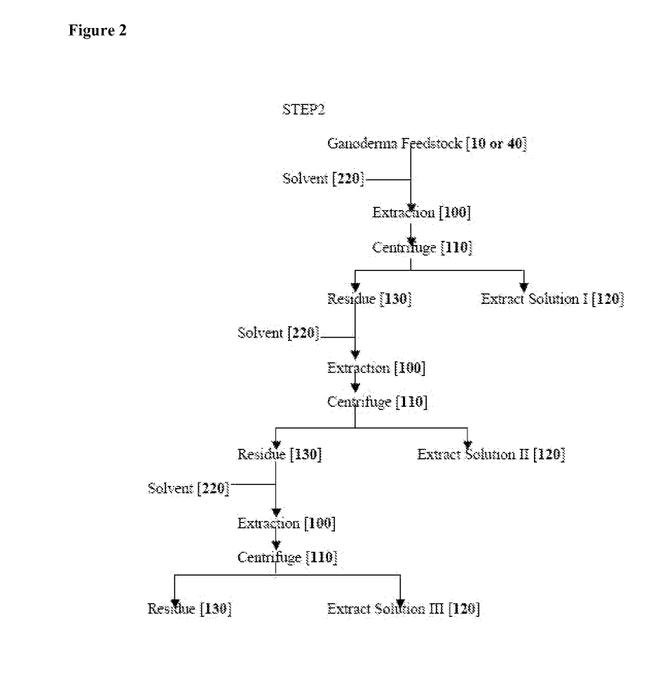

In one aspect, the present invention comprises extraction and concentration of the active triterpene compounds. A generalized description of this step is diagrammed in FIG. 2-Step 2. This Step 2 extraction process is a solvent leaching process. The feedstock for this extraction process is either the ganoderma species native feedstock [10] or the residue [40] following the SCCO2 extraction of the essential oil chemical constituents. The extraction solvent [220] may be aqueous ethanol, ethanol or other alcohol. In this method, the ganoderma species residue and the extraction solvent are loaded into an extraction vessel [100] and heated and stirred. It may be heated to 90° C., to about 80 ::C, to about 70° C., to about 60° C., or to about 60-80° C. The extraction is carried out for about 1-10 hours, for about 1-6 hours, for about 1-3 hours, or for about 2 hours. The resultant fluid-extract is centrifuged [120]. The filtrate (supernatant) is collected as product [120], measured for volume and solid content dry mass weight. The solid extraction residue material [130] is retained and saved for further processing (see Step 4). The extraction may be repeated as many times as is necessary or desired. It may be repeated 2 or more times, 3 or more times, 4 or more times, etc. For example, FIG. 1-STEP 2 shows a three-stage process, where the second stage and the third stage use the same methods and conditions. An example of this extraction step is found in Example 2 and the results in Tables 7-9.

TABLE 7
Comparison of triterpene content in ethanol leaching crude extract
and final purified triterpenoid extract composition.

| | | Purity (%) | | | |
|---|---|---|---|---|---|
| | | Ganoderic | Ganoderic | Ganodera- | Total |
| | yield | acid A | acid F | matriol | tritepenoid |
| Crude extract | 3.08 | 0.48 | 0.37 | 0.01 | 19.96 |
| Final product | 0.6 | 2.88 | 0.88 | 0.08 | 87.5 |

TABLE 8
HPLC analysis results of ganoderma ethanol leaching crude triterpene
extract fraction at concentration of 1.89 mg/ml in methanol.

| | | | | | Start | Stop | |
|---|---|---|---|---|---|---|---|
| | Retention | Area | Height | Width | time | time | Theoretical |
| ID | time (min) | (mAu · min) | (mAu) | (min) | (min) | (min) | plate |
| Ganoderic acid A | 13.216 | 48981 | 1806 | 3.02 | 12.93 | 15.95 | 306 |
| Ganoderic acid F | 21.557 | 23171 | 1468 | 0.32 | 21.37 | 21.69 | 72610 |
| Ganodermatiol | 34.347 | 30784 | 838 | 1.25 | 34.21 | 35.46 | 12080 |

TABLE 9
HPLC analysis results of ganoderma purified triterpene extract
fraction at concentration of 1.5 mg/ml in methanol.

| | | | | | Start | Stop | |
|---|---|---|---|---|---|---|---|
| | Retention | Area | Height | Width | time | time | Theoretical |
| ID | time (min) | (mAu · min) | (mAu) | (min) | (min) | (min) | plate |
| Ganoderic acid A | 13.259 | 318564 | 10951 | 1.02 | 12.43 | 13.45 | 2704 |
| Ganoderic acid F | 21.387 | 55473 | 2183 | 0.55 | 21.01 | 21.57 | 24193 |
| Ganodermatiol | 34.347 | 38020 | 3243 | 0.42 | 34.03 | 34.44 | 107004 |

Step 3. Purification of the Triterpene Fraction.

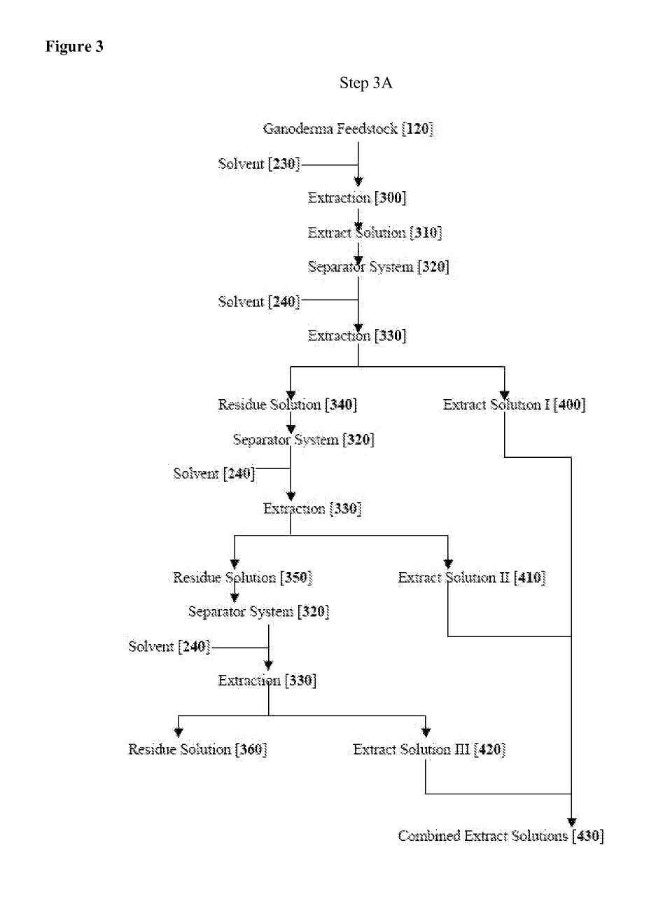

A generalized description of the extraction and purification of the triterpene fraction from the crude triterpene extracts of ganoderma species is diagrammed in FIG. 3-Step 3 (Appendix 1). The feedstock [120] is the crude triterpene extract from the three-stage ethanol leaching process of Step 2. The solvents are chloroform [230] and saturated sodium bicarbonate (NaHCO2) aqueous solution (10%) [240]. In this method, the crude triterpene extract feedstock []120 and the first extraction solvent [230] are loaded into an extraction vessel [100] and stirred to dissolve the crude triterpene fraction in the solvent. The chloroform solvent is introduced into a separator system [320]. Then, the second extraction solvent [240] is added to the solution in the separator system, mixed, vented, and allowed to stand for separation of the water based solvent (upper layer) from the chloroform solvent (lower layer). The water-based solution layer is collected [400], measured for volume and solid content dry mass weight. The chloroform (lower layer) residue solution [340] may be retained for further stages of NaHCO2 extraction. The NaHCO2 extraction may be repeated as many times as is necessary or desired. It may be repeated 2 or more times, 3 or more times, 4 or more times, etc. For example, FIG. 3-STEP 3A shows a NaHCO2 three-stage process, wherein the second stage and the third stage use the same methods and conditions. The water-based solutions collected from each extraction stage [400+410+420] are combined [430]. The combined solution is acidified. The acid is HCl [250]. The final pH of the solution may be about 3-5, or about 4. The acidified solution is then extracted [340] with the solvent chloroform [260] using a solvent separator system [320]. The chloroform solution layer containing the desired triterpenoids is collected and saved [450]. The chloroform extraction process may be repeated as many times as necessary or desired. For example, FIG. 3 STEP 3B shows a chloroform two-stage process, wherein the second stage uses the same methods and conditions. The water-based residue after completion of the extraction is discarded. The multi-stage chloroform solvent [480] is evaporated under reduced pressure using rotary evaporation and recycled [390]. The purified triterene fraction is dried [395] removing the remaining chloroform and saved as a purified triterpene fraction [500]. An example of this extraction step can be found in Example 3 and the results in Table 4.

The total yield of the purified triterpene fraction was 0.6% by mass weight based on the original ganoderma feedstock with a triterpene purity of about 88%, a 4-fold increase in purity from the crude triperpene extract fraction. Thus, the triterpenoid yield was greater than 65% of the triterpenoids present in the original ganoderma feedstock. The HPLC chromatograms reveal numerous unknown peaks which is expected given that greater than 130 highly oxygenated triterpenes and related compounds have been isolated from G. lucidum plant material. The total concentration of the three reference standards, ganoderic acid A, ganoderic acid F, and ganodermatriol, was about 4% supporting the importance of the total triterpenoid assay for quality control in commercial processing of a purified triterpene fraction.

Step 4. Water Leaching Process and Polysaccharide Precipitation

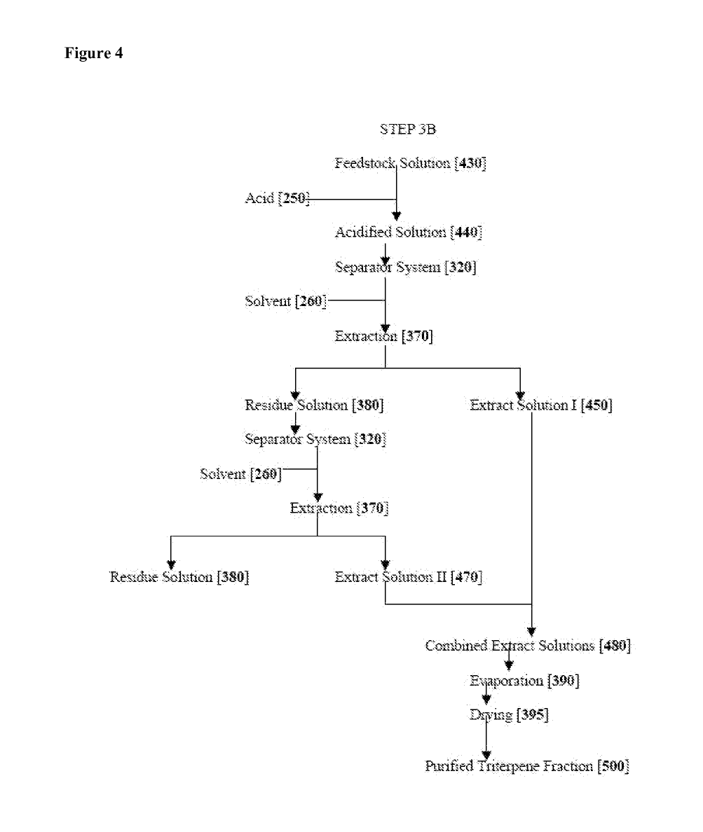

The polysaccharide extract fraction of the chemical constituents of ganoderma species has been defined in the scientific literature as the “water soluble, ethanol insoluble extraction fraction”. A generalized description of the extraction of the polysaccharide fraction from extracts of ganoderma species using water solvent leaching and ethanol precipitation processes is diagrammed in FIG. 4-Step 4. The feedstock [10] or [120] is the native ganoderma species plant material powder or the solid residue from the ethanol leaching extraction process of Step 2. This feedstock is leaching extracted in two stages. The solvent is distilled water [270]. In this method, the ganoderma species feedstock [10] or [120] and the extraction solvent [270] are loaded into an extraction vessel [700] and heated and stirred. It may be heated to 100° C., to about 60° C., or to about 70-80° C. The extraction is carried out for about 1-5 hours, for about 2-4 hours, or for about 2 hours. The extraction may be repeated as many times as necessary or desired. The multi-stage extraction solutions [700+720] are combined and the slurry is filtered [610], centrifuged [620], and the supernatant collected and evaporated [630] to remove water until an about 8-fold increase in concentration of the chemicals in solution [640]. Anhydrous ethanol [280] is then used to reconstitute the original volume of solution making the final ethanol concentration at 60-80% ethanol. A large precipitate [650] is observed. The solution is centrifuged [660], decanted [670] and the supernatant residue [750] may be saved for further processing or discarded. The precipitate product [740] after drying [680] is the purified polysaccharide fraction [760] that may be analyzed for polysaccharides using the colormetric method by using Dextran 5,000, 50,000, and 410,000 molecular weight as reference standards. The purity of the extracted polysaccharide fraction using 3 different molecular weight dextran as standards is about 80, 59, and 52%, respectively, with a total yield of 2% by % mass weight of the original native ganoderma feedstock. Combining the purity measures of the 3 dextran standards indicates a very high level of purity of greater than 95%. The principal impurity appears to be the desired lectin proteins (3% by mass weight) that also contain beneficial bioactive properties. The methods of the present invention are further taught in Example 4. The results are shown in Table 10. Moreover, AccuTOF-DART mass spectrometry was used to further profile the molecular weights of the compounds comprising the purified polysaccharide fraction. The results are shown in FIGS. 6 and 7.

TABLE 10
Polysaccharide analysis and protein analysis of water leaching extraction
and ethanol precipitation of the polysaccharide fraction.

| | Total | Dextran | Dextran | Dextran | Purity of | Protein |
|---|---|---|---|---|---|---|
| | yield | 5K (mg/ | 50K (mg/ | 410K (mg/ | protein | yield |
| | (%)* | mg pcp) | mg pcp) | mg pcp) | (%) | (%)* |
| Crude | 4.58 | | | | 1.48 | 0.074 |
| 60% pcp | 1.49 | 0.88 | 0.64 | 0.56 | 4.03 | 0.060 |
| 80% pcp | 1.99 | 0.80 | 0.59 | 0.52 | 2.99 | 0.059 |
| 95% pcp | 1.80 | 0.55 | 0.41 | 0.35 | 3.73 | 0.067 |

*Yields are % mass weight based on original ganoderma feedstock.

The ganoderma polysaccharide yield was about 2% by mass weight based on the original ganoderma plant feedstock. The purity of the polysaccharide fraction was 520-800 mg/g dextran standard equivalent indicating a purity of >90% ganoderma polysaccharide chemical constituents in the fraction. Based on a large number and variety of experimental approaches, it is quite reasonable to conclude that 2% yield is almost 100% of the water soluble-ethanol insoluble polysaccharides in the natural ganoderma species feedstock material. Furthermore, the principal impurity in the fraction appears to be the desired lectin proteins that make up about 3% mass weight of the purified polysaccharide fraction.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions from which alcohol was removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

Purity of the Extractions In performing the previously described extraction methods, it was found that a 50-99% yield by mass weight of the essential oil chemical constituents having greater than 95% purity of the essential oil chemical constituents in the original dried ganoderma bark feedstock of the ganoderma species can be extracted in the essential oil SCCO2 extract fraction (Step 1A). Using the methods as taught in Step 1B (SCCO2 Extraction and Fractionation Processes), the essential oil yield would be reduced due to the fractionation of the essential oil chemical constituents into highly purified (>90%) essential oil sub-fractions. In addition, the SCCO2 extraction and fractionation process as taught in this invention permits the ratios (profiles) of the individual chemical compounds comprising the essential oil chemical constituent fraction to be altered such that unique essential oil sub-fraction profiles can be created for particular medicinal purposes. For example, the concentration of the alcohol essential oil chemical constituents may be increased while simultaneous reducing the concentration of the fatty acid compounds or visa versa.

Using the methods as taught in Step 2 of this invention, an ethanol leaching crude triterpene fraction is achieved with a 3% yield by mass weight from the original ganoderma species feedstock having a 20% concentration of triterpene chemical constituents. This further equates to about a 66% yield of the triterpene related chemical constituents found in the native ganoderma species plant material.

Using the methods as taught in Step 3 of this invention (Purification of Triterpene Fraction), triterpene fractions with purities of greater than 85% by % dry mass of the extract may be obtained. It is possible to extract almost 100% of the triterpenes from the hydroalcoholic leaching extract feedstock. This equates to about 66% yield of the triterpene acid chemical constituents found in the native ganoderma species plant material.

Using the methods as taught in Step 4 of this invention, a purified polysaccharide fraction is achieved with a 1.5-2.0% mass weight yield from the original ganoderma species feedstock having a polysaccharide purity of greater than 90%. The polysaccharide yield is almost 100% of the water-soluble ethanol-insoluble polysaccharides present in the native ganoderma species feedstock material. The principle non-polysaccharide chemical constituents in this fraction appear to be the lectin proteins that make up about 3% by mass weight of the polysaccharide fraction. These proteins appear to act synergistically with the polysaccharides enhancing the beneficial bioactivity of the fraction.

Finally, the methods as taught in the present invention permit the purification (concentration) of the ganoderma species novel essential oil chemical constituent fractions, novel essential oil fractions or sub-fractions, a novel triterpene fraction, and a novel polysaccharide fraction to be as high as 99%% by mass weight of the desired chemical constituents in the essential oil fractions, as high as 87% by mass weight in the triterpene fraction, and as high as 95% by mass weight in the polysaccharide fraction. The specific extraction environments, rates of extraction, solvents, and extraction technology used depend on the starting chemical constituent profile of the source material and the level of purification desired in the final extraction products. Specific methods as taught in the present invention can be readily determined by those skilled in the art using no more than routine experimentation typical for adjusting a process to account for sample variations in the attributes of starting materials that is processed to an output material that has specific attributes. For example, in a particular lot of ganoderma species plant material, the initial concentrations of the essential oil chemical constituents, the triterpenes, and the polysaccharides are determined using methods known to those skilled in the art as taught in the present invention. One skilled in the art can determine the amount of change from the initial concentration of the essential oil chemical constituents, for instance, to the predetermined amounts or distribution (profile) of essential oil chemical constituents for the final extraction product using the extraction methods, as disclosed herein, to reach the desired concentration and/or chemical profile in the final ganoderma species extraction product.

Food and Medicaments

As a form of foods of the present invention, there may be formulated to any optional forms, for example, a granule state, a grain state, a paste state, a gel state, a solid state, or a liquid state. In these forms, various kinds of substances conventionally known for those skilled in the art which have been allowed to add to foods, for example, a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller, etc. may be optionally contained. An amount of the elderberry extract to be added to foods is not specifically limited, and for example, it may be about 10 mg to 5 g, preferably 50 mg to 2 g per day as an amount of take-in by an adult weighing about 60 kg.

In particular, when it is utilized as foods for preservation of health, functional foods, etc., it is preferred to contain the effective ingredient of the present invention in such an amount that the predetermined effects of the present invention are shown sufficiently.

The medicaments of the present invention can be optionally prepared according to the conventionally known methods, for example, as a solid agent such as a tablet, a granule, powder, a capsule, etc., or as a liquid agent such as an injection, etc. To these medicaments, there may be formulated any materials generally used, for example, such as a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller.

An administration amount of the effective ingredient (ganoderma extract) in the medicaments may vary depending on a kind, an agent form, an age, a body weight or a symptom to be applied of a patient, and the like, for example, when it is administrated orally, it is administered one or several times per day for an adult weighing about 60 kg, and administered in an amount of about 10 mg to 5 g, preferably about 50 mg to 2 g per day. The effective ingredient may be one or several components of the ganoderma extract.

The novel ganoderma species extractions may be administered daily, for one or more times, for the effective treatment of acute or chronic conditions. One method of the present invention comprises administering at least one time a day an extraction comprising ganoderma species constituent compounds. Methods also comprise administering such extractions more than one time per day, more than two times per day, more than three times per day and in a range from 1 to 15 times per day. Such administration may be continuously, as in every day for a period of days, weeks, months, or years, or may occur at specific times to treat or prevent specific conditions. For example, a person may be administered ganoderma species extracts at least once a day for years to enhance the immune system, or to prevent cardiovascular disease and stroke, or to prevent or treat inflammatory disorders and arthritis, or to treat hypertension, or to prevent and treat the common cold, influenza, or other viral diseases, or to prevent or treat bacterial diseases, or to treat diabetes mellitus, or to treat hyper-cholesterolemia, or to prevent or treat cancer.

The foregoing description includes the best presently contemplated mode of carrying out the present invention. This description is made for the purpose of illustrating the general principles of the inventions and should not be taken in a limiting sense. This invention is further illustrated by the following examples, which are not to be construed in any way as imposing limitations upon the scope thereof On the contrary, it is to be clearly understood that resort may be had to various other embodiments, modifications, and equivalents thereof, which, after reading the description herein, may suggest themselves to those skilled in the art without departing from the spirit of the present invention.

All terms used herein are considered to be interpreted in their normally accepted usage by those skilled in the art. Patent and patent applications or references cited herein are all incorporated by reference in their entireties.

### EXEMPLIFICATION

Materials and Methods

Botanicals

Red ganoderma lucidum (GL) dried mushrooms were obtained commercially. The active compounds concentration in feedstock were measured in-house and listed in Table 11.

TABLE 11
Chemical composition of ganoderma lucidum mushrooms.

| Chemicals | Weight % |
|---|---|
| Essential oil¹ | 1.2 |
| Tritepenoid² | 0.9 |
| Polysaccharide-glycoprotein³ | 1.59 |

¹Essential oil was estimated by highest yield of SCCO2 extraction at 70° C. and 500 bar.
²Tritepenoid was estimated by method extract.
³Polysaccharide-glycoprotein was estimated by water extract.

Organic Solvents

Acetone (CAS: 67-64-1), ≧99.5%, ACS reagent (179124); Acetonitrile (CAS: 75-05-8), for HPLC, gradient grade≧99.9% (GC) (000687); Hexane (CAS#: 110-54-3), 95+%, spectrophotometric grade (248878); Ethyl acetate (CAS#: 141-78-6), 99.5+%, ACS grade (319902); Ethanol (CAS: 64-17-5), denatured with 4.8% isopropanol (02853); Ethanol (CAS: 64-17-5), absolute, (02883), Methanol (CAS#: 67-56-1), 99.93%, ACS HPLC grade, (4391993); Chloroform (CAS#: 67-66-3), ≧99.0% (GC) and Water (CAS#: 7732-18-5), HPLC grade, (95304). All were purchased from Sigma-Aldrich.

Acids and Bases

Acetic acid (64-19-7), 99.7+%, ACS reagent (320099); Hydrochloric acid (7647-01-0), volumetric standard 1.0N solution in water (318949); Sodium bicarbonate (S263-1, Lot #: 037406) was purchased from Fisher Co. Bradford reagent (Product Number B 6916) was purchased from sigma.

Chemical Reference Standards

Serum albumin (9048-46-8), Albumin Bovine (BSA) Fraction V powder cell culture tested (A9418) was purchased from sigma; Ganoderic acid A (lot#: 07057-022), Ganoderic acid F (Lot#: 07068-037), and ganodermatriol (Lot#: 07060-128) were all purchased from sigma. Dextran standard 5000 (00269), 50, 000 (00891) and 410,000 (00895) certified according to DIN were purchased from fluka. The structures of these standards are shown in Table 12.

TABLE 12
Chemical structure of triterpenoid reference
standards for ganoderma lucidum.

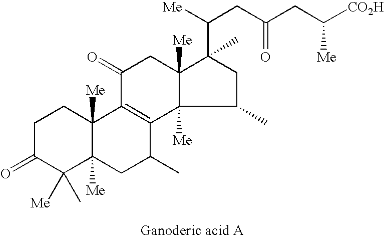

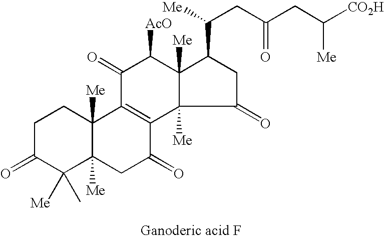

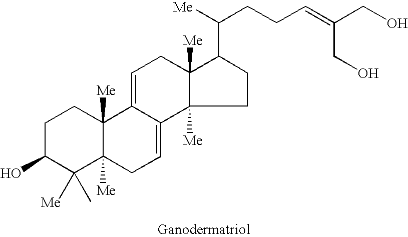

HPLC Method

Chromatographic system: Shimadzu high Performance Liquid Chromatographic LC-10AVP system equipped with LC10ADVP pump with SPD-M 10AVP photo diode array detector.

The ethanol extraction products obtained were measured on a reversed phase Jupiter C18 column (250<4.6 mm I. D., 5μ, 300 Å) (Phenomenex, Part #: 00G-4053-E0, serial No: 2217520-3, Batch No.: 5243-17). The injection volume was 10 μl and the flow rate of mobile phase was 1 ml/min. The column temperature was 25° C. The mobile phase consisted of A (2.5% aqueous acetic acid, v/v) and B (acetonitrile). The gradient was programmed as follows: with the first 12 minutes, B maintains at 30%, 12-30 min, solvent B increased linearly from 30% to 65%, and 30-40 min, B maintains at 65%, then 40-45 min, B linearly from 65% to 85%.

Methanol stock solutions of 3 standards list in Table 12 were prepared by dissolving weighted quantities of standard compounds into ethanol at 5 mg/ml. The mixed reference standard solution was then diluted step by step to yield a series of solutions at final concentrations of 2, 1, 0.5, 0.1, 0.05 mg/ml, respectively. All the stock solutions and working solution were used within 7 days and stored in +4° C. chiller and brought to room temperature before use. The solutions were used to identify and quantify the compounds in ganoderma lucidum extracts. Retention times of ganoderic acid A, ganoderic acid F and ganodermatriol were about 13.33, 21.63, and 34.42 min, respectively. A linear fit ranging from 0.01 to 20 μg was found. The regression equations and correlation coefficients were as follows: Ganoderic acid A: peak area=790642×C(μg)−23406, R²=0.9994 (N=6); Ganoderic acid F: peak area=513374×C(μg)−12458, R²=0.9999 (N=6); ganodermatriol: peak area=753902×C(μg)−29095, R²=0.9997 (N=6). HPLC results are shown in Table 13. The contents of the reference standards in each sample were calculated by interpolation from the corresponding calibration curves based on the peak area.

TABLE 13
HPLC analysis results of ganoderma lucidum triterpenoid reference
standards at concentration of 1 mg/ml in ethanol.

| | | | Peak | Peak | Start | Stop | |
|---|---|---|---|---|---|---|---|
| | Retention | Peak Area | Height | Width | time | time | Theoretical |
| ID | time (min) | (mAu · min) | (mAu) | (min) | (min) | (min) | plate¹ |
| Ganoderic | 13.333 | 2091756 | 70959 | 1.44 | 12.8 | 14.24 | 1371 |
| acid A | | | | | | | |
| Ganoderic | 21.632 | 1448041 | 80503 | 0.82 | 21.38 | 22.2 | 11134 |
| acid F | | | | | | | |
| Ganodermatiol | 34.421 | 2850919 | 153595 | 1.51 | 33.96 | 35.48 | 8314 |

¹Theoretical plates was calculated by: N = 16 × (t_{R}/w)². t_{R} is retention time and w is width of the peak, https://www.mn-net.com/web%5CMN-WEB-HPLCKatalog.nsf/WebE/GRUNDLAGEN.

GC-MS analysis

GC-MS analysis was performed at Shimadzu GCMS-QP2010 system. The system includes high-performance gas chromatograph, direct coupled GC/MS interface, electro impact (EI) ion source with independent temperature control, quadrupole mass filter et al. The system is controlled with GCMS solution Ver. 2 software for data acquisition and post run analysis. Separation was carried out on a Agilent J&W DB-5 fused silica capillary column (30 m−0.25 mm i.d., 0.25 μm film thickness) (catalog: 1225032, serial No: US5285774H) using the following temperature program. The initial temperature was 60° C., held for 2 min, then it increased to 120° C. at rate of 4° C./min, held for 15 min, then it increased to 200° C. at rate of 4° C./min, held for 15 min, then it increased to 240° C. at rate of 4° C./min, held for 15 min with total running time of 92 minutes. The sample injection temperature was 250° C. and 1 μl of sample was injected by auto injector at splitless mode in 1 minute. The carrier gas was helium and flow rate was controlled by pressure at 60 KPa. Under such pressure, the flow rate was 1.03 ml/min and linear velocity was 37.1 cm/min. MS ion source temperature was 230° C., and GC/MS interface temperature was 250° C. MS detector was scanned between m/z of 50 and 500 at scan speed of 1000 AMU/second. Solvent cutoff temperature was 3.5 min.

Rapid Quantification of Triterpenoids by Ultraviolet (UV) Spectrometry Method

Instrument: Shimazu UV-Vis spectrophotometer (UV 1700 with UV probe: S/N: A1102421982LP)
   Standards

Make triterpenoid standard Ganoderic acid F solution at concentration 0.2 mg/ml in saturated sodium bicarbonate (NaHCO₃). Dilute the solution to 0.2, 0.1, 0.05, 0.025, 0.0125 mg/ml with saturated sodium bicarbonate. Record the absorbance at 257 nm. The results are shown in Table 14.

TABLE 14
Rapid quantification of total triterpenoid by UV spectrometry
method using Ganoderic acid F as standards.

| | Ganoderic acid F | NaHCO₃ | Ganoderic acid F | Absorbance |
|---|---|---|---|---|
| Tube | solution (ml) | (ml) | (mg) | at 257 nm |
| Blank | 0 | 2 | 0 | 0 |
| S1 | 0.125 | 1.875 | 0.025 | 0.241 |
| S2 | 0.25 | 1.75 | 0.050 | 0.343 |
| S3 | 0.5 | 1.5 | 0.100 | 0.708 |
| S4 | 1 | 1 | 0.200 | 1.401 |
| S5 | 2 | 0 | 0.400 | 2.290 |

Polysaccharide Analysis (Dubois 1956)
Instrument:

Shimazu UV-Vis spectrophotometer (UV 1700 with UV probe: S/N: A1102421982LP)

Standard:

Colorimetric method has been used for polysaccharide analysis. Make 0.1 mg/ml stock dextran (Mw=5000, 50,000 and 410,000) solutions in distill water. Take 0.08, 0.16, 0.24, 0.32, 0.40 ml of stock solution and make up volume to 0.4 ml with distilled water. Then add in 0.2 ml 5% phenol solution and 1 ml concentrated sulfuric acid. The mixtures were allowed to stand for 10 minutes prior to performing UV scanning. The maximum absorbance was found at 488 nm. Then set the wavelength at 488 nm and measure absorbance for each sample. The results are shown in Table 15. The standard calibration curves were obtained for each of the dextran solutions as follows: Dextan 5K, Absorbance=0.01919+0.027782 C (μg), R²=0.97 (N=5); Dextan 50K, Absorbance=0.0075714+0.032196 C (μg), R²=0.96 (N=5); and Dextan 410K, Absorbance=0.03481+0.036293C (μg), R²=0.98 (N=5).

TABLE 15
Colorimetric analysis polysaccharide by
using Dextran as reference standard

| | Dextran | Distill | 5% | Sulfuric | Absorbance at 488 nm | | |
|---|---|---|---|---|---|---|---|
| | solution | water | phenol | acid | Mw = | Mw = | Mw = |
| Tube | (ml) | (ml) | (ml) | (ml) | 5K | 50K | 410K |
| blank | 0 | 0.40 | 0.2 | 1 | 0 | 0 | 0 |
| 1 | 0.08 | 0.32 | 0.2 | 1 | 0.238 | 0.301 | 0.335 |
| 2 | 0.16 | 0.24 | 0.2 | 1 | 0.462 | 0.504 | 0.678 |
| 3 | 0.24 | 0.16 | 0.2 | 1 | 0.744 | 0.752 | 0.854 |
| 4 | 0.32 | 0.08 | 0.2 | 1 | 0.907 | 1.045 | 1.247 |
| 5 | 0.40 | 0.00 | 0.2 | 1 | 1.098 | 1.307 | 1.450 |

Polysaccharide Molecular Weight Analysis

Polysaccharide molecular weight analysis was on HPLC system equipped with a RID-10A refractive index detector. The flow-rate was set at 0.6 ml/min. The analyses were performed using a 300×7.8 mm I. D. TSK-GEL G4000PW_{XL} column (10 μm particle size, 300 Å pore size, Tosoh Corporation, Minato-ku, Tokyo, Japan. Catalog No: 08022, Column No: H3463). The mobile phase was distilled water and the injection volume was 10 μl. The column temperature was 35° C. and RID cell temperature was 40° C. The analysis time was 40 min.

Distill water stock solutions of different molecular weight of Dextran standards were prepared by dissolving weighted quantities of standard compounds into distilled water at concentration of 5 mg/ml. Retention times of dextran 5 k, dextran 25 k, dextran 50 k, dextran 270 k and dextran 410 k were about 15.70, 13.82, 12.93, 11.08 and 10.76 min, respectively, shown in Table 16. A linear curve fit was obtained by plotting retention time (X axis) vs. Log Mw (Y axis). The regression equation was: Log (Mw)=9.669−0.3817×Rt (R²=0.99859). The unknown samples molecular weight can be calculated by above equation by knowing sample's retention time.

TABLE 16
HPLC-RID analysis results of Dextran referemce standards.

| | | | | | | | Peak | | | |
|---|---|---|---|---|---|---|---|---|---|---|
| | | | Ret. | | Peak Area | | Height | | Start | Stop |
| | | Log | Time | Peak | Percent | Peak | Percent | Peak | Time | Time |
| Name | M_{w} | (M_{w}) | (min) | Area | (%) | Height | (%) | Width | (min) | (min) |
| Dextran | 410000 | 5.6 | 15.7 | 536282 | 98.8 | 5999 | 94.4 | 4.62 | 12.9 | 17.5 |
| 5K | | | | | | | | | | |
| Dextran | 270000 | 5.4 | 13.8 | 555103 | 94.3 | 6266 | 81.4 | 4.43 | 11.9 | 16.4 |
| 25k | | | | | | | | | | |
| Dextran | 50000 | 4.7 | 12.9 | 457221 | 91.6 | 4758 | 72.0 | 4.46 | 11.0 | 15.5 |
| 50K | | | | | | | | | | |
| Dextran | 25000 | 4.4 | 11.1 | 439369 | 78.4 | 4444 | 46.4 | 4.57 | 9.2 | 13.8 |
| 270k | | | | | | | | | | |
| Dextran | 5000 | 3.7 | 10.8 | 366093 | 71.6 | 3487 | 36.7 | 4.78 | 9.1 | 13.8 |
| 410K | | | | | | | | | | |

Direct Analysis in Real Time (DART) Mass Spectrometry
Instruments

JOEL AccuTOF DART LC time of flight mass spectrometer (Joel USA, Inc., Peabody, Mass., USA). This Time of Flight (TOF) mass spectrometer technology does not require any sample preparation and yields masses with accuracies to 0.00001 mass units.

Methods for Fraction Analysis

The instrument settings utilized to capture and analyze fractions are as follows: For cationic mode, the DART needle voltage is 3000 V, heating element at 250° C., Electrode 1 at 100 V, Electrode 2 at 250 V, and helium gas flow of 7.45 liters/minute (L/min). For the mass spectrometer, orifice 1 is 10 V, ring lens is 5 V, and orifice 2 is 3 V. The peaks voltage is set to 600 V in order to give resolving power starting a approximately 60 m/z, yet allowing sufficient resolution at greater mass ranges. The micro-channel plate detector (MCP) voltage is set at 2450 V. Calibrations are performed each morning prior to sample introduction using a 0.5 M caffeine solution standard (Sigma-Alrich Co., St. Louis, USA). Calibration tolerances are held to ≦5 mmu.

The samples are introduced into the DART helium plasma with sterile forceps ensuring that a maximum surface area of the sample is exposed to the helium plasma beam. To introduce the sample into the beam, a sweeping motion is employed. This motion allows the sample to be exposed repeatedly on the forward and back stroke for approximately 0.5 sec/swipe and prevented pyrolysis of the sample. This motion is repeated until an appreciable Total Ion Current (TIC) signal is observed at the detector, then the sample is removed, allowing for baseline/background normalization.

For anionic mode, the DART and AccuTOF MS are switched to negative ion mode. The needle voltage is 3000 V, heating element 250° C., Electrode 1 at 100 V, Electrode 2 at 250 V, and helium gas flow at 7.45 L/min. For the mass spectrometer, orifice 1 is −20 V, ring lens is −13 V, and orifice 2 is −5 V. The peak voltage is 200 V. The MCP voltage is set at 2450 V. Samples are introduced in the exact same manner as cationic mode. All data analysis is conducted using MassCenterMain Suite software provided with the instrument.

### EXAMPLE 1

Example of Step 1A: Single Step SFE Maximal Extraction and Purification of Ganoderma Essential Oil Fraction.

Experiments were performed using a SFT 250 purchased from Supercritical Fluid Technologies, Inc. (Newark, Del.) that is designed for pressures and temperatures up to 690 bar and 200° C., respectively. This apparatus allows simple and efficient extractions at supercritical conditions with flexibility to operate in either dynamic or static modes. This apparatus consists of mainly three modules; an oven, a pump and control, and collection module. The oven has one preheat column and one 100 ml extraction vessel. The pump module is equipped with a compressed air-driven pump with constant flow capacity of 300 ml/min. The collection module is a glass vial of 40 ml, sealed with caps and septa for the recovery of extracted products. The equipment is provided with micrometer valves and a flow meter. The extraction vessel pressure and temperature are monitored and controlled within ⊥3 bar and ⊥1° C.

5 grams of ground red young Lingzhi fruit powder with size above 105 μm sieved measured using a screen (140 mesh) were loaded into a 100 ml extraction vessels for each experiment. Glass wool was placed at the two ends of the column to avoid any possible carry over of solid material. The oven was preheated to the desired temperature before the packed vessel was loaded. After the vessel was connected into the oven, the extraction system was tested for leakage by pressurizing the system with CO₂ (˜850 psig), and purged. The system was closed and pressurized to the desired extraction pressure using the air-driven liquid pump. The system was then left for equilibrium for ˜3 min. A sampling vial (40 ml) was weighed and connected to the sampling port. The extraction was started by flowing CO₂ at a rate of ˜5 SLPM (10 g/min), which is controlled by a meter valve. The yield was defined to be the weight ratio of total exacts to the feed of raw material. The yield was defined as the weight percentage of the oil extracted with respect to the initial charge of the raw material in the extractor. A full extraction design was adopted varying the temperature from 40-80° C. and from 100-500 bar.

### EXAMPLE 2

Example of Step 2: Ethanol Leaching Extraction of Crude Triterpene Fraction.

A typical example of a 3 stage solvent extraction of the triterpene chemical constituents of ganoderma species is as follows: The feedstock was 25 gm of ground ganoderma species fruit body SFE residue from Step 1 SCCO2 extraction of the essential oil (40° C., 300 bar). The solvent was 500 ml of ethanol. In this method, the feedstock material and 500 ml ethanol were separately loaded into 1000 ml extraction vessel and mixed in a heated water bath at 70° C. for 2 hours. The extraction solution was filtered using Fisherbrand P4 filter paper having a particle retention size of 4-8 μm, centrifuged at 2000 rpm for 10 minutes. The filtrate (supernatant) was collected for yield calculation and HPLC analysis. The particulate residue of Stage 1 was extracted for 2 hours (Stage 2) and the residue from Stage 2 was extracted for 2 hours using the aforementioned methods. The supernatant fluid-extracts from the 3-stage extractions were combined and the ethanol evaporated and recycled using reduced pressure rotary evaporation. The extract was vacuum dried at 50° C. for 12 hours. The dried crude triterpene extract fraction was measured for mass balance, total triterpene content using a total triterpenoid assay and analyzed using HPLC. The final residue from the 3-stage extraction was collected and saved for further extraction (see below).

The total yield of the 3-stage ethanol leaching process crude triterpene extract was about 3% by mass weight based on the original ganoderma species feedstock with a total triterpenoid purity of about 20%. To achieve greater purity of the triterpene chemical constituents, additional processing is required (see Example 3).

### EXAMPLE 3

Example of Step 3. Triterpene Fraction Purification.

A typical experimental example of purification of the triterpenes in the crude ethanol leaching fraction is as follows: 1 g of the ethanol leaching crude triterpene fraction of Step 2 was dissolved in 50 ml of chloroform and stirred for 5 min in an extraction vessel at room temperature. This clear solution was poured into a 200 ml separator funnel. 40 ml of saturated NaHCO₃ (10%) aqueous solution is added to the chloroform solution. This mixture was vigorously shaken for 15 sec, the pressure released, and shaken vigorously a second time for 15 sec. Less than 30 sec of total mixing was sufficient to allow the solutes to come to equilibrium between the chloroform phase and the water based solution phase. Special care must be taken to vent the pressure as a large volume of C0₂ was produced during this process. The separator funnel is allowed to stand undisturbed until the two solution layers become clearly separated (about 30 min). The stopcock of the separator funnel is then opened the lower chloroform layer drained into separate flask and saved for two additional NaHCO₃ solvent extractions. The remaining water based solution is poured from the top of the funnel and saved. Two additional stages of NaHCO3 extracted of the chloroform solution were performed using the same methods. The three stage NaHCO₃ extract solutions (120 ml) were combined and acidified using 6N HCl to a pH of 4 (about 3 ml). The acidified solution was poured into a clean 200 ml separator funnel. 50 ml of chloroform was introduced into the separator funnel in two stages to extract the triterpene compounds from the acidified water based solution. The methods were as described above at room temperature. The two chloroform layers were collected, combined, and saved. The remainder water based solution was discarded. The combined chloroform solution containing the purified triterpene chemical constituents was evaporated under reduced pressure using rotary evaporation and the chloroform recycled. The purified triterpene extraction fraction was oven dried at 50° C. removing the remaining chloroform. The yield was calculated by mass balance, total triterpene content using a total triterpenoid UV spectrometry assay, and analyzed using HPLC.

### EXAMPLE 4

Example of Step 4 Polysaccharide Fraction Extraction.

A typical experimental example of solvent extraction and precipitation of the water soluble, ethanol insoluble purified polysaccharide fraction chemical constituents of ganoderma species is as follows: The feedstock was the solid residue from the 25 gm Step 1 SFE extraction and Step 2 ethanol leaching extraction. The feedstock was extracted using 500 ml of distilled water for two hours at 70° C. in two stages. The two extraction solutions were combined and the slurry was filtered using Fisherbrand P4 filter paper (pore size 4-8 =82 m) and centrifuged at 2,000 rpm for 20 minutes. The supernatant was collected. Rotary evaporation was used to concentrate the clear supernatant extract solution from 1000 ml to 200 ml. Then, 600 or 800 ml of anhydrous ethanol was added to make up a final ethanol concentration of 60 or 80%. The solution was allowed to sit for 1 h and a precipitate was observed. The extraction solution was centrifuged at 2,000 rpm for 20 minutes and the supernatant decanted and either saved for further processing or discarded. Mass balance was performed before and after precipitation to calculate the yield of polysaccharides and proteins. The precipitate was collected and dried in an oven at 50° C. for 12 hours. The dried polysaccharide fraction was weighed and dissolved in water for analysis of polysaccharide and protein purity using a colormetric method with dextran as reference standards and the Bradford protein assay, respectively.

### EXAMPLE 5

The following ingredients are mixed for the formulation

| | | |
|---|---|---|
| Extract of G. lucidum fruit body | 150.0 | mg |
| Essential Oil Fraction (10 mg, 6.6% dry weight) | | |
| Polyphenolic Fraction (120 mg, 80% dry weight) | | |
| Polysaccharides (40 mg, 26.6% dry weight) | | |
| Stevioside (Extract of Stevia) | 12.5 | mg |
| Carboxymethylcellulose | 35.5 | mg |
| Lactose | 77.0 | mg |
| Total | 275.0 | mg |

The novel extract of ganoderma species comprises an essential oil fraction, triterpene fraction, and polysaccharide fraction by % mass weight greater than that found in the natural ganoderma species plant material or conventional extraction products. The formulations can be made into any oral dosage form and administered daily or to 15 times per day as needed for the physiological, psychological, and medical effects (immune enhancement, diabetes mellitus, anti-platelet aggregation and anti-thrombosis, cardiovascular and cerebrovascular disease prevention and treatment, anti-atherosclerosis, anti-hypercholesterolemia, anti-hypertension, anti-inflammatory, anti-allergic, anti-arthritis, anti-rheumatic, anti-auto immune diseases, anti-viral including, but not limited to, the common cold, influenza, HIV, herpes simplex, herpes zoster, and hepatitis B, anti-bacterial, and cancer prevention and therapy).

### EXAMPLE 6

The following ingredients were mixed for the following formulation

| | | |
|---|---|---|
| Extract of G. lucidum fruit body | 150.0 | mg |
| Essential Oil Fraction (30 mg, 20% dry weight) | | |
| Polyphenolic Fraction (60 mg, 40% dry weight) | | |
| Polysaccharides (60.0 mg, 40% dry weight) | | |
| Vitamin C | 15.0 | mg |
| Sucralose | 35.0 | mg |
| Mung Bean Powder 10:1 | 50.0 | mg |
| Mocha Flavor | 40.0 | mg |
| Chocolate Flavor | 10.0 | mg |
| Total | 300.0 | mg |

The novel extracts of ganoderma species comprises an essential oil, triterpene, and polysaccharide chemical constituent fractions by % mass weight greater than that found in the natural plant material or conventional extraction products. The formulation can be made into any oral dosage form and administered safely up to 15 times per day as needed for the physiological, psychological and medical effects desired (see Example 1, above).

### REFERENCES CITED

1. Wang Y Y et al. Bioorg Med Chem 10: 1057-1062, 2002.
2. Sone Y et al. Agric Biol Chem 49:2641-2653, 1985.
3. Bao X et al. Carbohydr Res 336:127-140, 2001.
4. Bao X et al. Phytochemistry 59:175-181, 2002.
5. Van Der Horn L et al. Transplantation 60:438-443, 1995.
6. Miyasaka N et al. Biochem Biophy Res Commun 186:385-390, 1992.
7. Streeper R T & Satsangi N. SUPELCO 14:1-3, 1995.
8. Giner-Larza E M et al. J Ethnopharmacol 73:61-69, 2000.
9. Wachtel-Galor S et al. In: Herbal and Traditional Medicine. Molecular Aspects of Health (Packer L, Ong C N, Halliwell B, eds), Marcel Dekker, New York, 2004, pp 179-228.
10. Kohda H et al. Chem Pharm Bull 33:1367-1374, 1985.
11. Lee J M et al. Phytother Res 15:245-249, 2001.
12. Zhu M et al. Phytother Res 13:529-531, 1999.
13. Shimizu A et al. Chem Pharm Bull 33:3012-3015, 1985.
14. Su C Y et al. Thromb Res 9:135-145, 2000.
15. Jong S C & Birmingham J M. Adv Appl Microbiol 37:101-134, 1992.
16. Lee S Y et al. Chem Pharm Bull 38:1359-1364, 1990.
17. Kammatsuse K et al. Yakugaku Zasshi 105:942-947, 1985.
18. Komoda Y et al. Chem Pharm Bull 37:531-533, 1989.
19. Shieh Y H et al. Am J Chin Med 29:501-507, 2001.
20. Park E J et al. Biol Pharm Bull 20:417-420, 1997.
21. Eo S K et al. J Ethnopharmacol 68:129-136, 1999.
22. Kim Y S et al. J Ethnopharmacol 72:451-458, 2000.
23. Oh K W et al. J Ethnopharmacol 72-221-227, 2000.
24. El-Mekkawy S et al. Phytochemistry 49:1651-1657, 1998.
25. Nostro A et al. Lett Appl Microbiol 30:379-384, 2000.
26. Wu B S et al. J Nat Prod 64:1121-1122,2001,
27. Zhang Q H et al. Int J Mushrooms 1:207-215, 1999.
28. Lu H et al. Oncol Rep 8:1341-1345, 2001,
29. Bradford M. Anal. Biochem 72:248-254, 1976.
30. Dubois M et al. Analytical Chem 28:350-356, 1956.
31. Pharmacopoeia of the People's Republic of China. Part II. The State Pharmacopoeia Committee of the People's Republic of China, Beijing Chemical Industry Press, 200.

We claim:

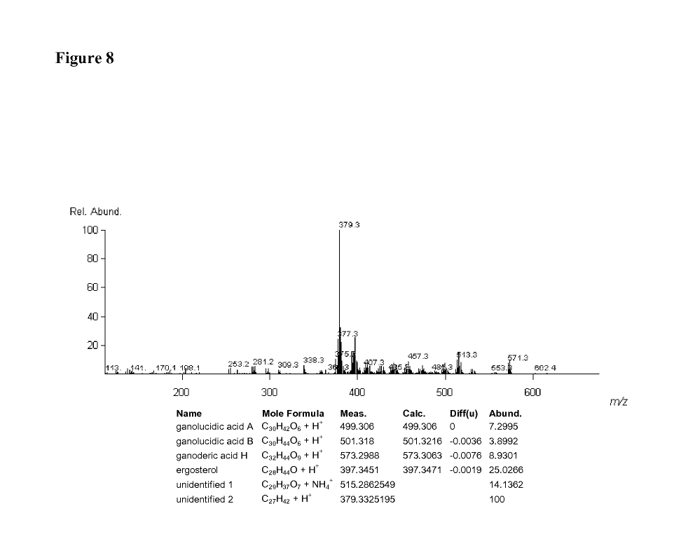

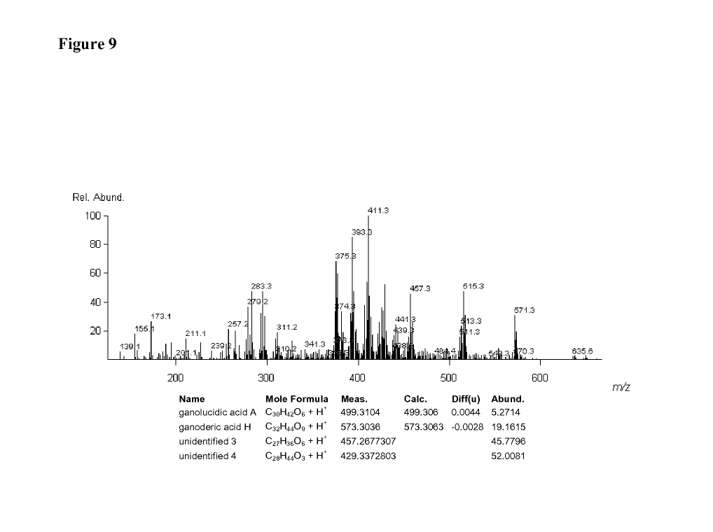

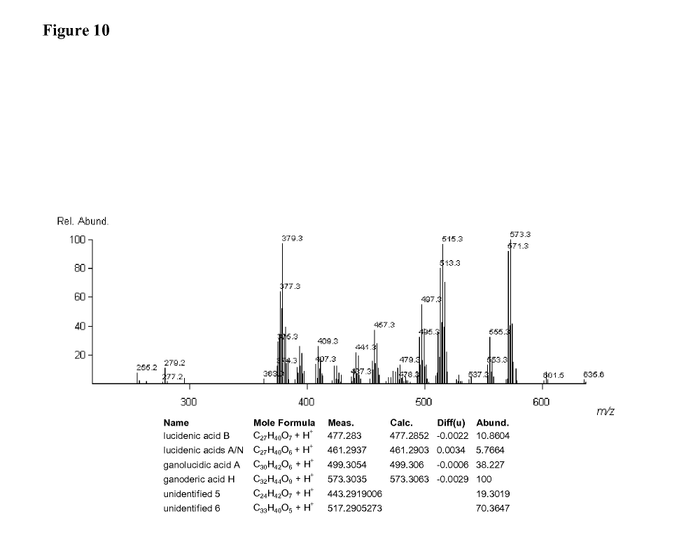

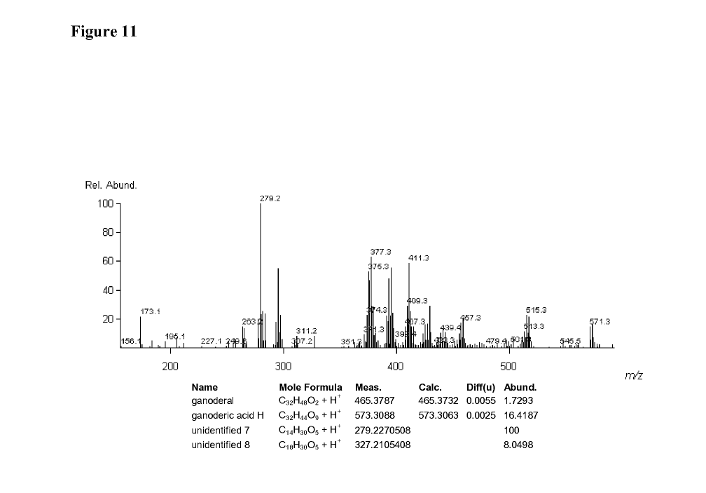

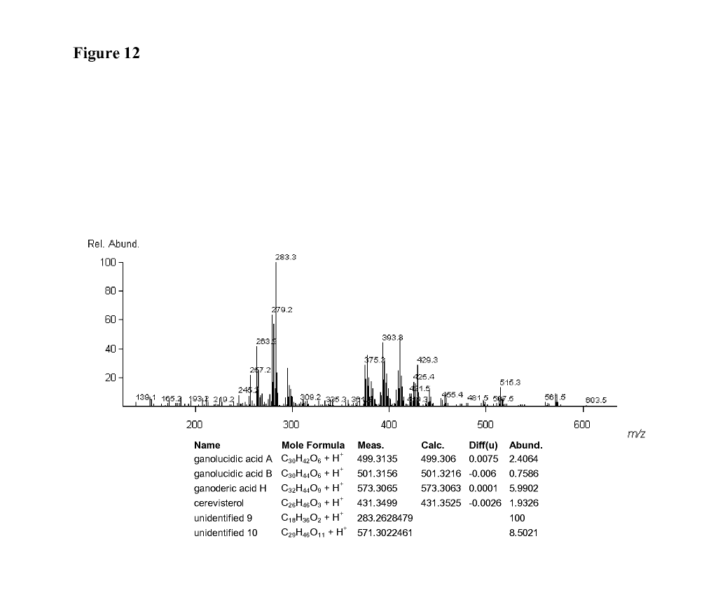

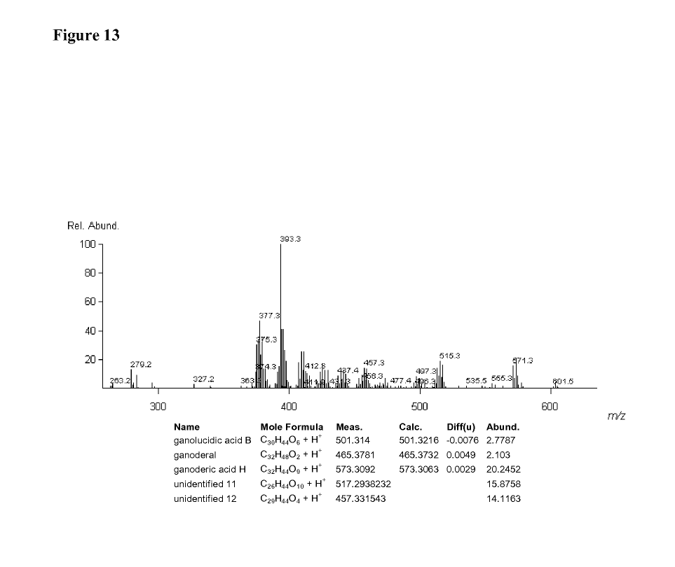

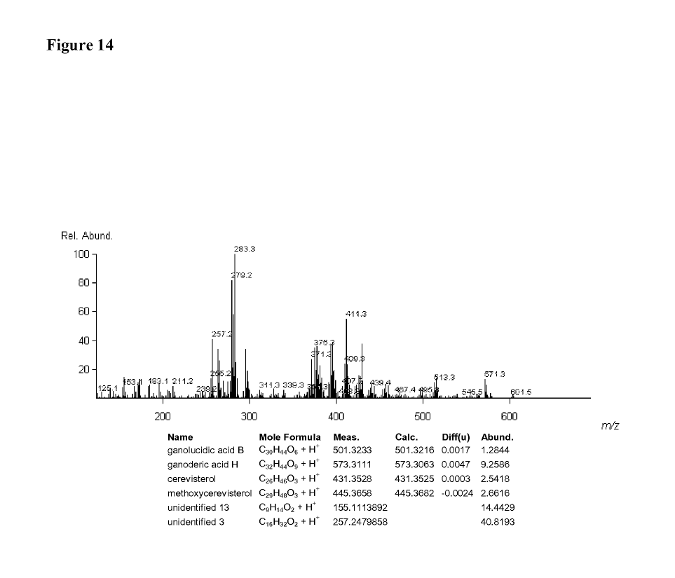

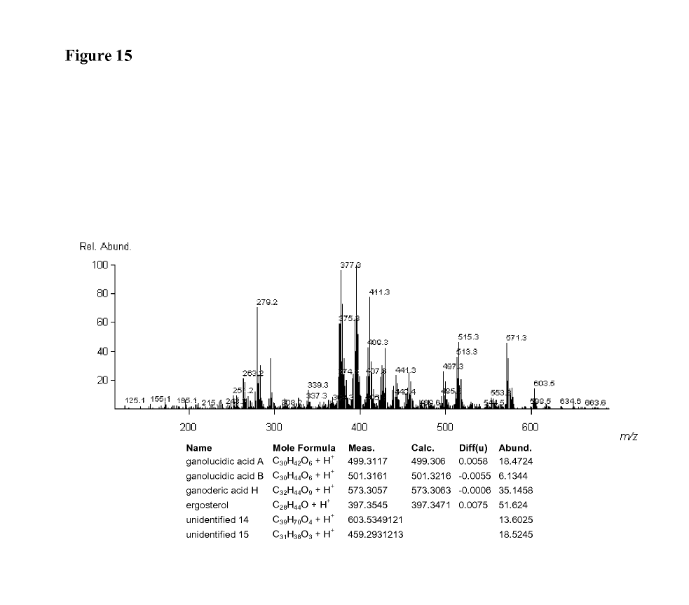

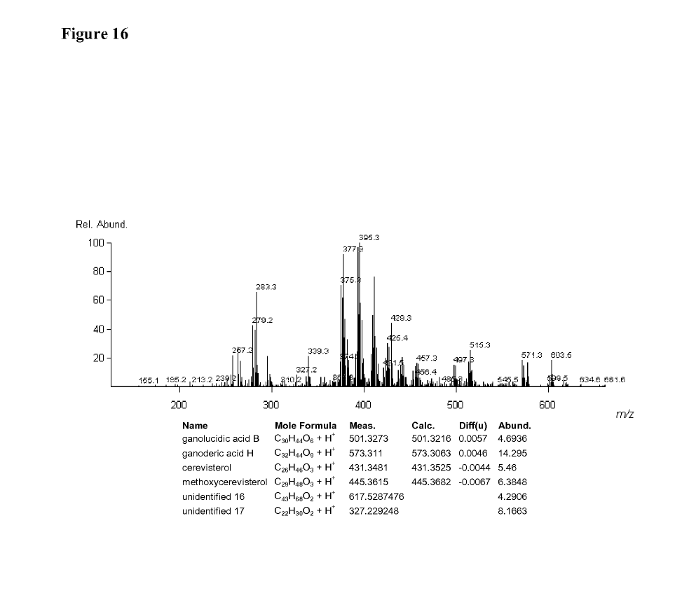

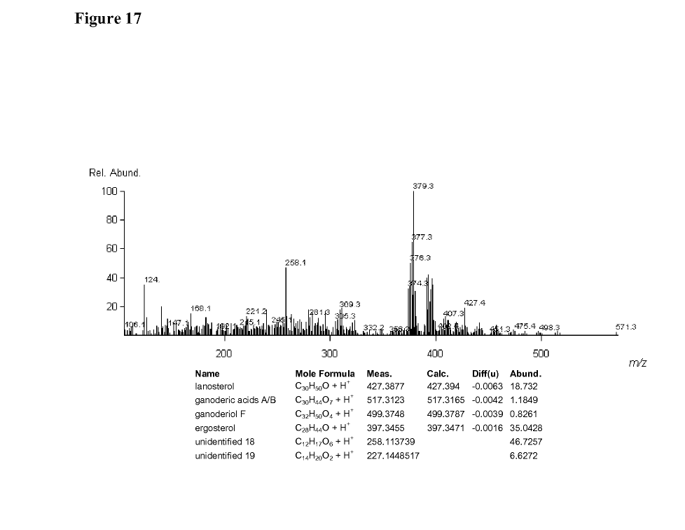

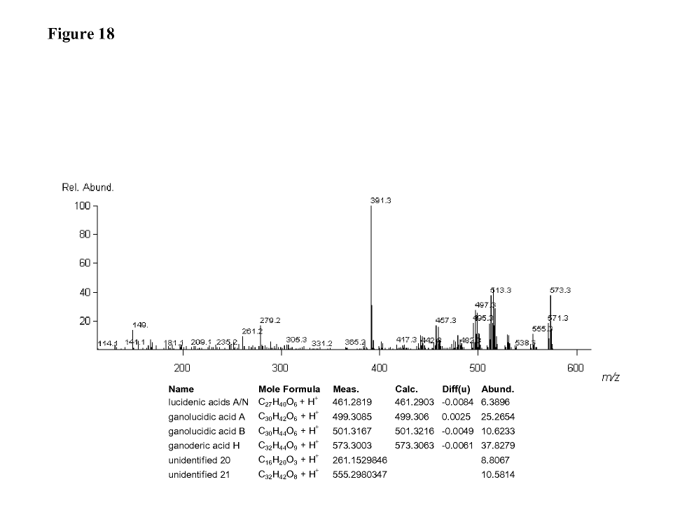

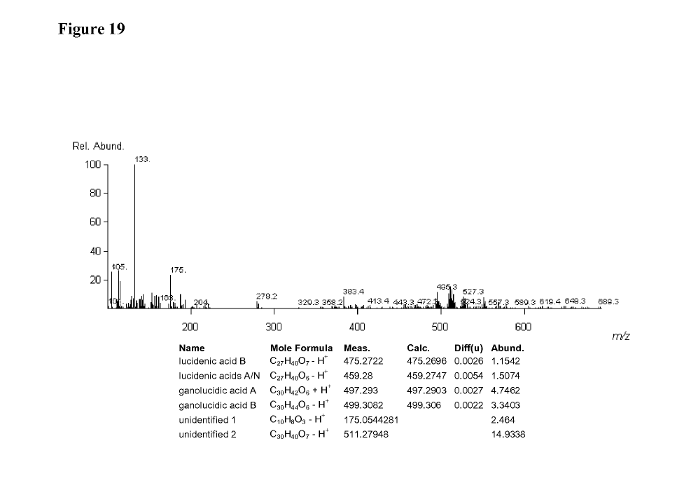

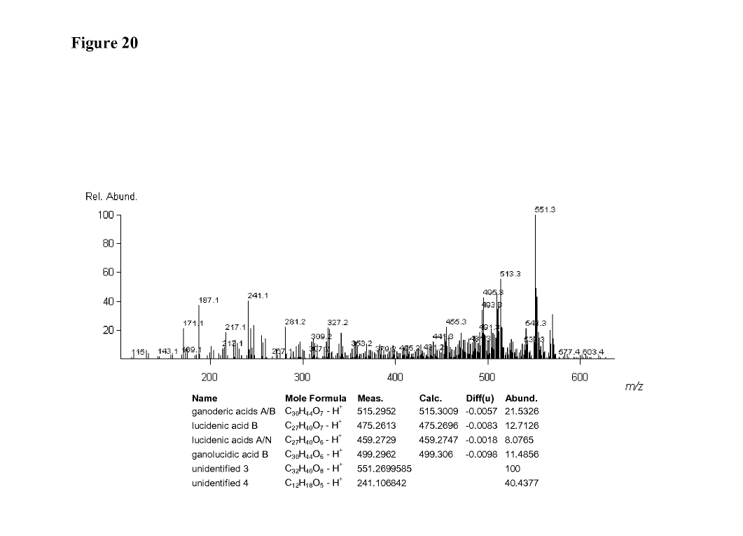

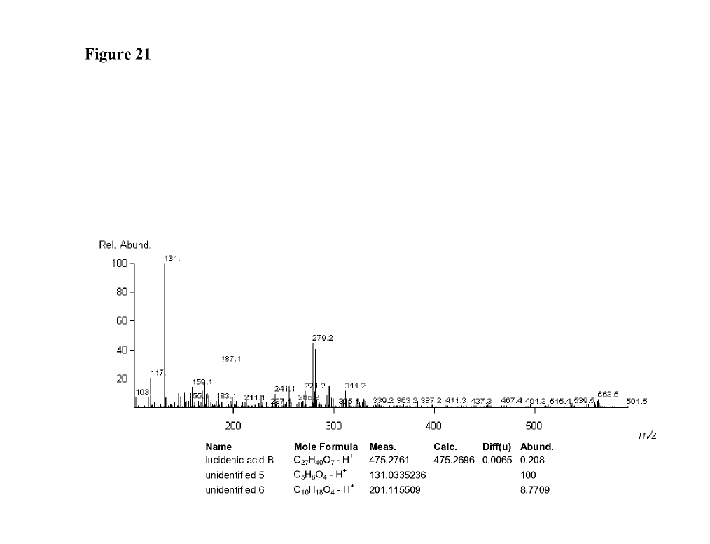

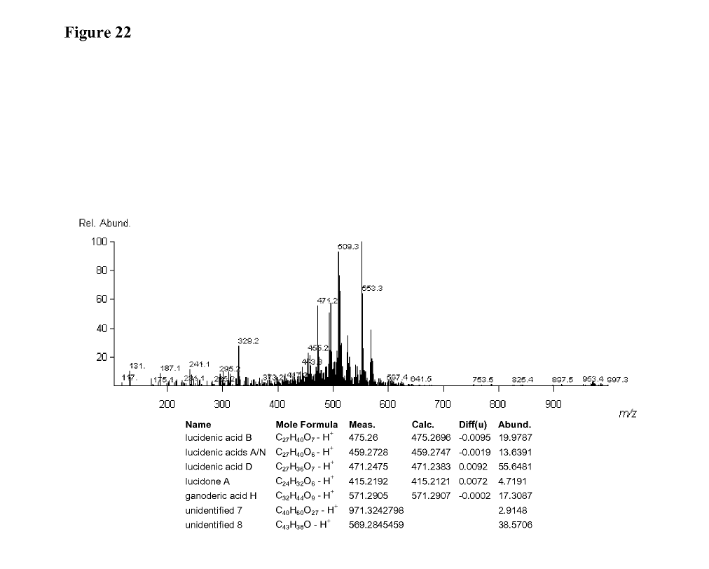

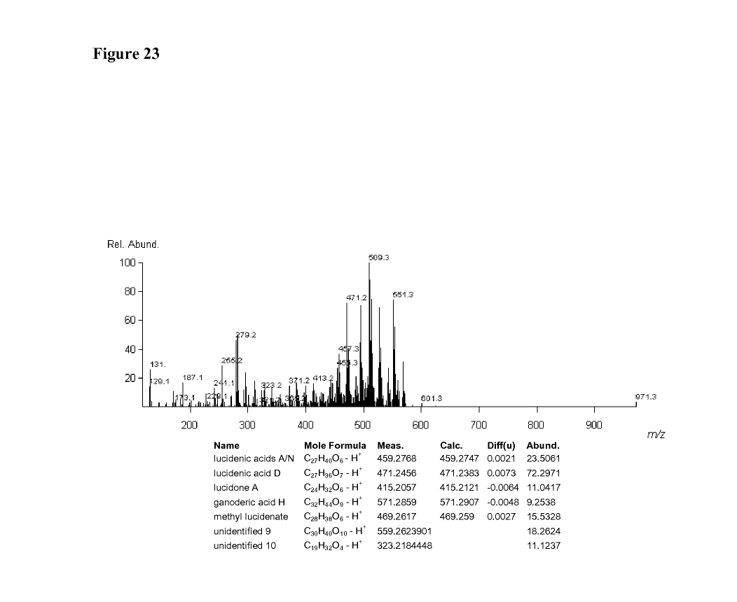

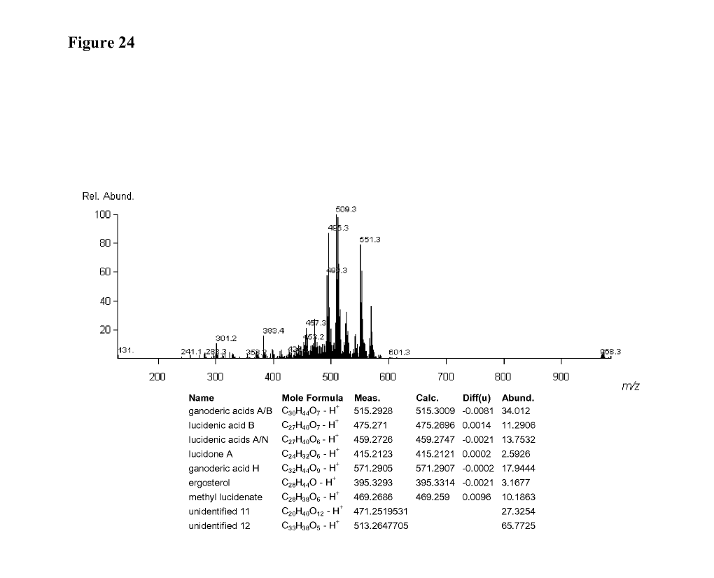

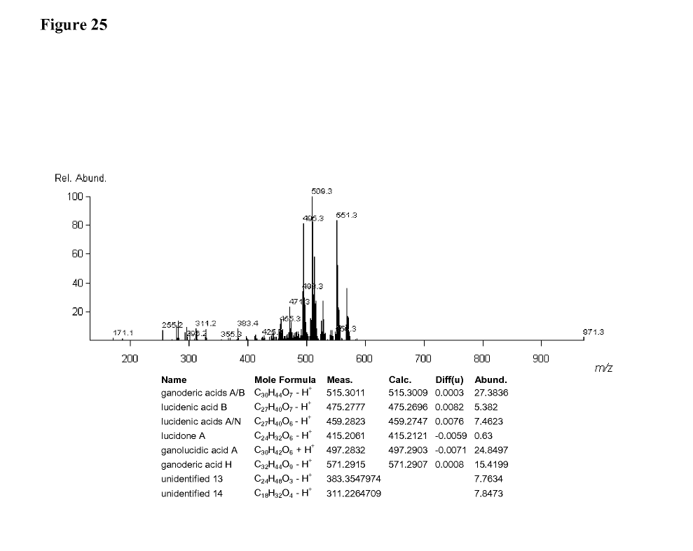

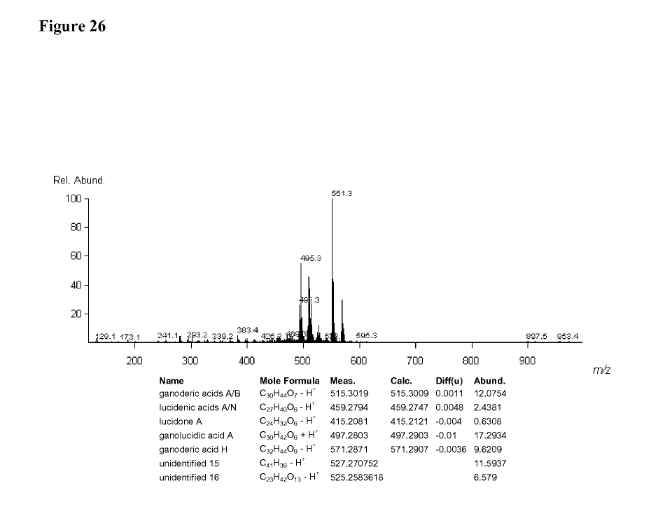

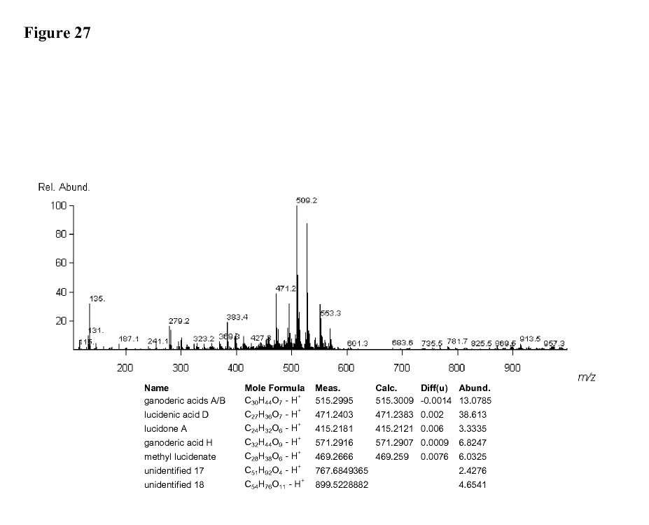

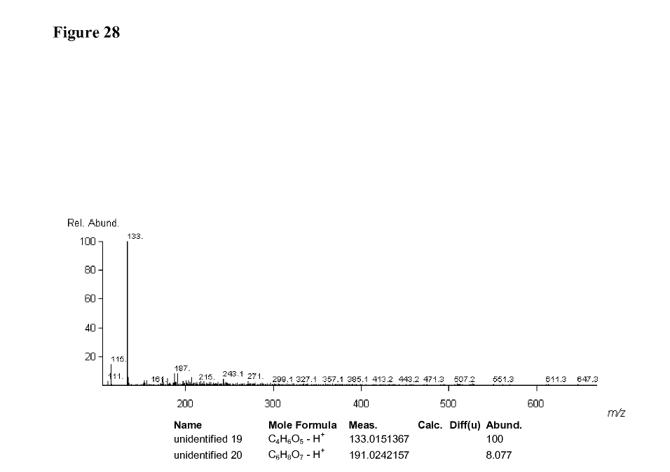

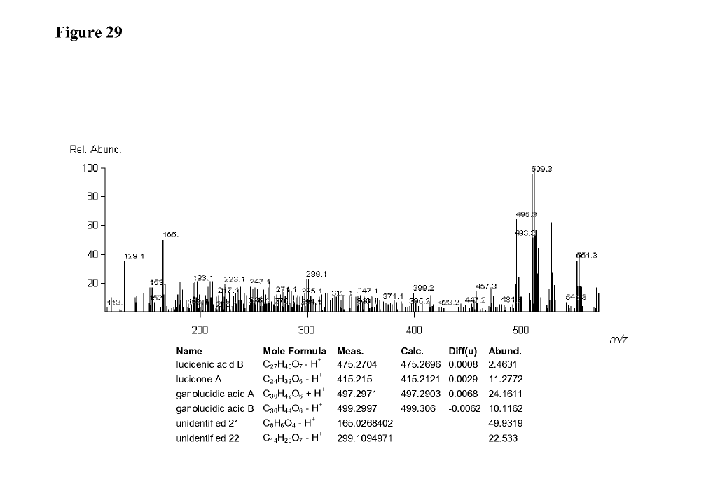

1 A ganoderma species extract comprising a fraction having a Direct Analysis in Real Time (DART) mass spectrometry chromatogram of any of FIGS. 6 to 29.

2. The ganoderma species extract of claim 1, wherein the extract comprises a compound selected from the group consisting of an essential oil, a triterpene, a polysaccharide, and combinations thereof.

3. The ganoderma species extract of claim 2, wherein the essential oil is selected from the group consisting of 9,12-octadecadienoic acid, linoelaidic acid, n-hexadecanoic acid, octoanoic acid, tetradecanoic acid, pentadecanoic acid, 9-octadecenoic acid, octadecanoic acid, 2-propenoic acid, tridecyl ester, 1-undecanol, 1-dodecanol, 1-tetradecanol, 1-hexadecanol, 1-heptadecanol, 1-eicosanol, and combinations thereof.

4. The ganoderma species extract of claim 2, wherein the triterpene is selected from the group consisting of ganoderic acid, lucidenic acid, ganolucidic acid, ganoderiol, lucidone, lucidumol, ganodermenonol, ganodermadiol, ganodermatriol, ganodermanondiol, ganodermanontriol, and combinations therof.

5. The ganoderma species of claim 2, wherein the polysaccharide is selected from the group consisting of glucose, arabinose, galactose, rhamnose, xylose uronic acid and combinations thereof.

6. The ganoderma species of claim 2, wherein the amount of essential oil is greater than 8% by weight.

7. The ganoderma species extract of claim 2, wherein the amount of essential oil is from 25% to 90% by weight.

8. The ganoderma species extract of claim 2, wherein the amount of essential oil is from 50% to 90% by weight.

9. The ganoderma species extract of claim 2, wherein the amount of essential oil is from 75% to 90% by weight.

10. The ganoderma species extract of claim 2, wherein the amount of triterpene is greater than 2% by weight.

11. The ganoderma species extract of claim 2, wherein the amount of triterpene is from 25% to 90% by weight.

12. The ganoderma species extract of claim 2, wherein the amount of triterpene is from 50% to 90% by weight.

13. The ganoderma species extract of claim 2, wherein the amount of triterpene is from 75% to 90% by weight.

14. The ganoderma species extract of claim 2, wherein the amount of polysaccharide is greater than 15% by weight.

15. The ganoderma species extract of claim 2, wherein the amount of polysaccharide is from 25% to 90% by weight.

16. The ganoderma species extract of claim 2, wherein the amount of polysaccharide is from 50% to 90% by weight.

17. The ganoderma species extract of claim 2, wherein the amount of polysaccharide is from 75% to 90% by weight.

18. The ganoderma species extract of claim 1, wherein the extract comprises an essential oil from 2% to 99% by weight, a triterpene from 5% to 88% by weight, and a polysaccharide from 2% to 95% by weight.

19. Food or medicament comprising the ganoderma species extract of claim 1.

20. A method of preparing a ganoderma species extract having at least one predetermined characteristic comprising sequentially extracting a ganoderma species plant material to yield an essential oil fraction, a triterpene fraction, and a polysaccharide fraction by
a) extracting a ganoderma species plant material by super critical carbon dioxide extraction to yield an essential oil fraction and a first residue;
b) extracting the first residue from step a) by alcoholic extraction to yield the triterpene fraction and a second residue; and
c) extracting the second residue from step b) by water extraction and precipitating the polysaccharide with alcohol to yield the polysaccharide fraction.

21. The method of claim 20, wherein step a) comprises:
1) loading in an extraction vessel ground ganoderma species plant material;
2) adding carbon dioxide under supercritical conditions;
3) contacting the ganoderma species plant material and the carbon dioxide for a time; and
4) collecting an essential oil fraction in a collection vessel.

22. The method of claim 20, further comprising the step of altering the essential oil chemical compound ratios by fractionating the essential oil fraction with a supercritical carbon dioxide fractional separation system.

23. The method of claim 21, wherein supercritical conditions comprise 60 bars to 800 bars of pressure at 35° C. to 90° C.

24. The method of claim 21, wherein supercritical conditions comprise 60 bars to 500 bars of pressure at 40° C. to 80° C.

25. The method of claim 21, wherein the time is 30 minutes to 2.5 hours.

26. The method of claim 21, wherein the time is 1 hour.

27. The method of claim 20, wherein step b) comprises:
1) contacting the first residue from step a) with an alcoholic solvent for a time sufficient to extract triterpene chemical constituents;
2) purifying the triterpene chemical constituents using liquid-liquid solvent extraction processes.

28. The method of claim 27, wherein one solvent is chloroform and the other solvent is a saturated NaHCO₃ aqueous solution.

29. The method of claim 27, wherein the alcoholic solvent is ethanol.

30. The method of claim 27, wherein step 1) is carried out at 30° C. to 100° C.

31. The method of claim 27, wherein step 1) is carried out at 60° C. to 100° C.

32. The method of claim 27, wherein the time is 1-10 hours.

33. The method of claim 27, wherein the time is 1-5 hours.

34. The method of claim 27, wherein the time is 2 hours.

35. The method of claim 20, wherein step c) comprises:
1) contacting either ganoderma species plant material or the second residue from step b) with water for a time sufficient to extract polysaccharides; and
2) precipitating the polysaccharides from the water solution by alcohol precipitation.

36. The method of claim 35, wherein the water is at 70° C. to 90° C.

37. The method of claim 35, wherein the water is at 80° C. to 90° C.

38. The method of claim 35, wherein the time is 1-5 hours.

39. The method of claim 35, wherein the time is 2-4 hours.

40. The method of claim 35, wherein the time is 2 hours.

41. The method of claim 35, wherein the alcohol is ethanol.

42. A ganoderma species extract prepared by the method of claim 20.

43. A ganoderma species extract comprising ergosterol, ganolucidic acid A at 25 to 35% by weight of the ergosterol, ganolucidic acid B at 10 to 20% by weight of the ergosterol, and ganoderic acid H at 30 to 40% by weight of the ergosterol.

44. A ganoderma species extract comprising ganoderic acid H and ganolucidic acid A at 25 to 35% by weight of the ganoderic acid H.

45. A ganoderma species extract comprising ganoderic acid H, lucidenic acid B at 5 to 15% by weight of the ganoderic acid H, lucidenic acids A/N at 1 to 10% by weight of the ganoderic acid H, and ganolucidic acid A at 35 to 45% by weight of the ganoderic acid H.

46. A ganoderma species extract comprising ganoderic acid H and ganoderal at 5 to 15% by weight of the ganoderic acid H.

47. A ganoderma species extract comprising ganoderic acid H, ganolucidic acid A at 35 to 45% by weight of the ganoderic acid H, ganolucidic acid B at 10 to 20% by weight of the ganoderic acid H, and cerevisterol at 30 to 40% by weight of the ganoderic acid H.

48. A ganoderma species extract comprising ganoderic acid H, ganolucidic acid B at 10 to 20% by weight of the ganoderic acid H, and ganoderal at 5 to 15% by weight of the ganoderic acid H.

49. A ganoderma species extract comprising ganoderic acid H, ganolucidic acid B at 10 to 20% by weight of the ganoderic acid H, methoxycerevisterol at 20 to 30% by weight of the ganoderic acid H, and cerevisterol at 20 to 30% by weight of the ganoderic acid H.

50. A ganoderma species extract comprising ergosterol, ganolucidic acid A at 30 to 40% by weight of the ergosterol, ganolucidic acid B at 5 to 15% by weight of the ergosterol, and ganoderic acid H at 65 to 75% by weight of the ergosterol.

51. A ganoderma species extract comprising ganoderic acid H, ganolucidic acid B at 30 to 40% by weight of the ganoderic acid H, methoxycerevisterol at 40 to 50% by weight of the ganoderic acid H, and cerevisterol at 35 to 45% by weight of the ganoderic acid H.

52. A ganoderma species extract comprising ergosterol, ganolucidic acids A/B at 1 to 10% by weight of the ergosterol, ganoderiol F at 1 to 10% by weight of the ergosterol, and lanosterol at 50 to 60% by weight of the ergosterol.

53. A ganoderma species extract comprising ganoderic acid H, ganolucidic acid A at 60 to 70% by weight of the ganoderic acid H, ganolucidic acid B at 25 to 35% by weight of the ganoderic acid H, and lucidenic acids A/N at 10 to 20% by weight of the ganoderic acid H.
